(19) United States
(12) Patent Application Publication
(10) Pub. No.: US 2017/0276659 A1
(43) Pub. Date: Sep. 28, 2017

(54) SYSTEMS AND METHODS FOR ASSESSING THERAPEUTIC PROTEINS

(71) Applicant: Biogen MA Inc., Cambridge, MA (US)

(72) Inventors: Iva Turyan, Waltham, MA (US); Nikhil Khatwani, Medford, MA (US); Zoran Sosic, Cambridge, MA (US)

(73) Assignee: Biogen MA Inc., Cambridge, MA (US)

(21) Appl. No.: 15/508,551

(22) PCT Filed: Sep. 4, 2015
(86) PCT No.: PCT/US15/48590

§ 371 Date: Mar. 3, 2017

Related U.S. Application Data
(60) Provisional application No. 62/046,446, filed on Sep. 5, 2014.

(51) Int. Cl. G01N 33/15; G01N 27/416

(52) U.S. Cl. CPC G01N 33/15; G01N 27/4168; G01N 27/4161

(57) ABSTRACT

Systems and methods for assessing the oxidation status of a pharmaceutical preparations are provided herein. Methods provided herein are particularly useful for assessing pharmaceutical preparations comprising therapeutic proteins (e.g., therapeutic antibodies).

### RELATED APPLICATION

This application claims the benefit under 35 U.S.C. §119(e) of U.S. provisional application No. 62/046,446, filed Sep. 5, 2014, which is incorporated by reference herein in its entirety.

### BACKGROUND

Pharmaceutical preparations comprising therapeutic proteins are major products of the biotechnology industry and have gained importance in the treatment of a broad number of diseases for which no other therapy is available. Instability, however, represents a serious problem in the development of such pharmaceutical preparations. Oxidation is a major degradation pathway for constituents of pharmaceutical preparations, such as active protein therapeutics, and can be promoted by light exposure, transition metals, peroxide from or induced by excipients or by the presence of oxygen during the manufacturing and storage process. Therefore, it is desirable to analyze potential oxidation changes during such processes. Current methods for quantifying the degree of protein oxidation include costly and time consuming techniques such as mass spectrometry (LC-MS) by generating peptide maps. Currently, there is no cost effective high throughput analytical tool in place for oxidative analysis of therapeutic proteins.

### SUMMARY

Aspects of the disclosure generally relate to systems and methods for assessing pharmaceutical preparations based on redox potentiometric measurements (e.g., using indirect potentiometry). Systems and methods provided herein are useful for assessing lot-to-lot variation in the manufacture of pharmaceutical preparations, which is advantageous for minimizing or eliminating significant differences in effective doses being administered and occurrence of unexpected side effects. Methods provided herein are particularly useful for assessing pharmaceutical preparations comprising therapeutic proteins (e.g., therapeutic antibodies). Accordingly, in some embodiments, methods provided herein may be implemented as quality control steps in manufacturing processes for therapeutic proteins and related compositions which, in contrast with certain small molecules, can be a challenge to control because of innate variability of biological systems and reactivity of amino acid side chains.

According to some aspects of the disclosure, potentiometric methods provided herein are useful for assessing oxidation status of pharmaceutical preparations comprising therapeutic proteins. In some embodiments, methods provided herein involve indirect potentiometry. With convention techniques, protein oxidation is typically measured using focused peptide mapping analysis (e.g., using LC-MS), which is a time consuming technique (2-3 days for sample preparation and data analysis). In contrast, potentiometric methods provided herein facilitate rapid assessment of protein oxidation status (e.g., ˜15 minutes), and utilize instrumentation which is relatively cost effective, portable and easy to operate. Methods provided herein also offer advantages of very low sample consumption and short analysis time.

Furthermore, due to the complexity and time consuming nature of conventional techniques, high throughput analyses of protein oxidation has not be attainable. In contrast, systems and methods provided herein enable high-throughput oxidation analysis of proteins and other molecules. While potentiometric measurements disclosed herein may be related to oxidation status, in some embodiments, potentiometric measurements are related to other aspects of a molecule, including for example, activity, stability, quality, size, conformation, etc.

In some embodiments, methods provided herein are based on detection of changes in potential between indicator and reference electrodes caused by alterations in a solution. In some embodiments, the detected changes can be related to one or more properties (e.g., oxidation status) of a molecule under analysis. In some embodiments, alterations are associated with a reaction between a protein (and/or other molecule) and a redox couple in the solution. In some embodiments, potentiometric changes resulting from such a reaction are relatable to one or more properties (e.g., oxidation status) of a molecule under analysis. In some embodiments, an initial potential of a redox couple may be corrected based on a change in potential caused by a one or more excipients, buffer components or inactive ingredients of pharmaceutical preparation, such that potential changes resulting from an active ingredient (e.g., a therapeutic protein) can be detected. In some embodiments, quantitation is performed using a standard addition based method, which is useful for eliminating matrix effects. In some embodiments, percentage oxidation of a standard is determined from a focused molecular mapping analysis (e.g., using mass spectroscopy) and related to potentiometric measurements of the standard, thereby producing a standard curve. In such embodiments, the standard curve may be used to determine oxidation status of a test molecule based on potentiometric measurements.

In some embodiments, indirect potentiometric methods are provided herein which are based on detection of a change in potential of a redox couple as a result of its interaction with oxidized species in a solution. In some embodiments, a change in potential is measured following addition of i) formulation buffer (FB), ii) an unknown sample and/or iii) one or more standard(s) (e.g., two standards) to a redox solution. In some embodiments, an initial potential of a redox couple is corrected based on a change in potential caused by a one or more excipients, buffer components or inactive ingredients of a pharmaceutical preparation (which may be referred to herein as a formulation buffer correction). In some embodiments, an unknown sample is added and a change in potential is measured. In some embodiments, quantitation of oxidation status involves use of a standard addition method using one or more additions of the reference standard (e.g., two standard additions). In some embodiments, oxidation status (e.g., percent oxidation) of a reference standard is pre-determined from a focused peptide mapping analysis (e.g., using mass spectrometry for a particular protein under study; a total percentage oxidation is determined as the sum of the % oxidation of each peptide of that protein from a focused peptide map analysis by mass spectrometry). In some embodiments, a change in potential of a redox couple corrected for matrix inference (ΔE) after addition of the sample and standard(s) is plotted vs a total amount of protein over known % oxidation (μg/% oxidation). In some embodiments, percent oxidation is calculated by the following relation:

percent (%) oxidation=μg/X,

in which X is determined using a linear fit and interpolating a graph to determine the value of X (abscissa) at Y (ordinate)=0.

Aspects of the disclosure relate to methods of assessing a pharmaceutical preparation. In some embodiments, the methods involve potentiometrically determining the oxidation status of the pharmaceutical preparation. In some embodiments, the methods comprise determining a redox potential of the pharmaceutical preparation; and determining the oxidation status of the pharmaceutical preparation based on the redox potential of the pharmaceutical preparation. In some embodiments, the step of determining the redox potential of the pharmaceutical preparation comprises determining a potential of a redox couple corrected by a formulation buffer of the pharmaceutical preparation. In some embodiments, the oxidation status of the pharmaceutical preparation is determined based on a change in potential of the redox couple due to its interaction with the pharmaceutical preparation. In some embodiments, methods provided herein further comprise (i) determining a potential between an indicator electrode and a reference electrode disposed in a solution that comprises a redox couple, thereby determining the redox potential; (ii) determining a change in potential between the indicator electrode and the reference electrode resulting from presence of the pharmaceutical preparation in the solution that comprises the redox couple; and (iii) determining the oxidation status of the pharmaceutical preparation based on the change in potential. In some embodiments, methods provided herein further comprise (i) determining a potential between an indicator electrode and a reference electrode disposed in a solution that comprises a redox couple, thereby determining the redox potential; (ii) determining a potential between an indicator electrode and a reference electrode resulting from presence of the formulation buffer in a solution that comprises a redox couple, thereby correcting the redox potential; (iii) determining a change in potential between the indicator electrode and the reference electrode resulting from presence of the pharmaceutical preparation in the solution that comprises the redox couple and formulation buffer; and (iv) determining the oxidation status of the pharmaceutical preparation based on the change in potential. In some embodiments, step (iv) comprises evaluating a reference standard that relates a change in potential to an oxidation status of the pharmaceutical preparation.

In some embodiments, the reference standard relates the change in potential to a mass spectroscopically determined oxidation status of the pharmaceutical preparation. In some embodiments, the indicator electrode is a platinum, gold, palladium, rhodium, or carbon electrode. In some embodiments, the reference electrode is a silver/silver chloride reference electrode. In some embodiments, the redox couple comprises Hexamamineruthenium(II)chloride and Hexamamineruthenium(III)chloride. In some embodiments, the concentration of Hexamamineruthenium(II)chloride is between 10-2 M and 10-4 M. In some embodiments, the concentration of Hexamamineruthenium(III)chloride is between 10-2 M and 10-4 M. In some embodiments, the concentration of Hexamamineruthenium(II)chloride is 10-2 M and the concentration of Hexamamineruthenium(III)chloride is 10-4 M. In some embodiments, the pharmaceutical preparation comprises a protein, a nucleic acid, or a small molecule. In some embodiments, the pharmaceutical preparation comprises a protein. In some embodiments, the protein is an immunoglobulin or fragment thereof. In some embodiments, the pharmaceutical preparation comprises STX-100, natalizumab, BIIB037, Anti-TWEAK, Anti-BDCA2, Daclizumab. In some embodiments, the pharmaceutical preparation comprises an excipient. In some embodiments, the excipient comprises a polysorbate or an amino acid. In some embodiments, the polysorbate is selected from the group consisting of polysorbate 20, polysorbate 40, polysorbate 60, and polysorbate 80. In some embodiments, the amino acid comprises arginine, glycine or histidine. In some embodiments, the change in potential measured in step (ii) is adjusted based on an extent of change in potential between the indicator electrode and the reference electrode that results from a buffer component of the pharmaceutical preparation. In some embodiments, the buffer component comprises sodium sulfate, sodium citrate, sodium phosphate, succinate, sodium chloride, potassium nitrate, or sucrose. In some embodiments, the methods further comprise, prior to step (i), removing a component of the pharmaceutical preparation that affects the potential of the redox couple. In some embodiments, the component is an arginine or a polysorbate. In some embodiments, the oxidation status is indicative of activity of the pharmaceutical preparation. Thus, in some embodiments, the methods further comprise evaluating activity of the pharmaceutical preparation using a bioassay. In some embodiments, the bioassay comprises evaluating efficacy or toxicity of the pharmaceutical preparation. In some embodiments, the oxidation status of the pharmaceutical preparation is determined on two or more occasions. In some embodiments, the methods further comprise determining a change in activity of the pharmaceutical preparation based on the oxidation status of the pharmaceutical preparation is determined on the two or more occasions.

Further aspects of the disclosure relate to methods of assessing activity of a biomolecule. In some embodiments, the methods comprise determining a redox potential between an indicator electrode and a reference electrode disposed in a solution comprising the biomolecule; and determining activity of the biomolecule based on the redox potential. In some embodiments, the methods comprise (i) determining a potential between an indicator electrode and an reference electrode disposed in a solution that comprises a redox couple; (ii) determining a change in potential between the indicator electrode and the reference electrode resulting from presence of the biomolecule in the solution that comprises the redox couple; and (iii) determining the activity of the biomolecule based on the change in potential. In some embodiments, step (iii) comprises evaluating a reference standard that relates a change in potential to the activity of the biomolecule. In some embodiments, the biomolecule is a protein, nucleic acid, or small molecule. In some embodiments, the protein is an immunoglobulin or fragment thereof. In some embodiments, the indicator electrode is a platinum, gold, palladium, rhodium, or carbon electrode. In some embodiments, the reference electrode is a silver/silver chloride or saturated calomel reference electrode. In some embodiments, the redox couple comprises Hexamamineruthenium(III)chloride and Hexamamineruthenium(II)chloride. In some embodiments, the concentration of Hexamamineruthenium(II)chloride is between 10-2 M and 10-4 M. In some embodiments, the concentration of Hexamamineruthenium(III)chloride is between 10-2 M and 10-4 M. In some embodiments, the concentration of Hexamamineruthenium(II)chloride is 10-2 M and the concentration of Hexamamineruthenium(III)chloride is 10-4 M.

Further aspects of the disclosure relate to systems for assessing the oxidation status of a pharmaceutical preparation. In some embodiments, the systems comprise (i) a container configured for housing a solution; (ii) an indicator electrode and a reference electrode; (iii) a potential measuring device operably connectable to the indicator electrode and reference electrode and configured for obtaining one or more potential measurements between the indicator electrode and the reference electrode; and (iv) a computer operably connectable to the potential measuring device. In some embodiments, the computer is configured for determining the oxidation status of the pharmaceutical preparation based on one or more potential measurements obtained from the potential measuring device while the pharmaceutical preparation is present in the solution. In some embodiments, the indicator electrode and the reference electrode are both disposable in the container. In some embodiments, the solution comprises a redox couple. In some embodiments, the computer is configured for determining the oxidation status of the pharmaceutical preparation based on a change in potential between the indicator electrode and the reference electrode resulting from presence of the pharmaceutical preparation in the solution that comprises the redox couple. In some embodiments, the computer comprises an input interface configured to receive information from the potential measuring device indicative of one or more potential differences measured between the indicator electrode and reference electrode. In some embodiments, the computer comprises at least one processor programmed to evaluate a model that relates the one or more potential differences to the oxidation status of the pharmaceutical preparation. In some embodiments, the computer comprises an output interface configured to output a signal indicative of the oxidation status. In some embodiments, the computer is configured to determine the oxidation status of a protein based on a reference standard that relates potential differences to a mass spectrometrically determined oxidation status of the pharmaceutical preparation.

### BRIEF DESCRIPTION OF DRAWINGS

FIG. 1 is a non-limiting example of a process flow diagram for determining the oxidation of proteins using indirect potentiometric analysis;

FIGS. 2A and 2B provided a non-limiting example of a standard addition method;

FIG. 3 is a non-limiting example of data generated from testing 65 mg/mL of STX-100 (RS012-001) in formulation buffer (10 mM Sodium Citrate 5% (w/v) sucrose, pH 6.1);

FIG. 4 is a non-limiting example of data generated from testing 25 mg/mL of Tysabri (NB-1143-113) in formulation buffer (10 mM Sodium Phosphate, 140 mM NaCl, pH 6.1);

FIG. 5 is a non-limiting example of data generated from testing 51.7 mg/mL of BART (17506-7-1) in formulation buffer (10 mM Sodium Citrate, 150 mM L-Arginine, 0.05% PS 80, pH 6.3);

FIG. 6 is a non-limiting example of data generated from testing 100 mg/mL of Tweak (RS030-002) in formulation buffer (10 mM Sodium Succinate, 150 mM L-Arginine, pH 5.5);

FIG. 7 is a non-limiting example of data generated from testing 48.5 mg/mL of Anti-BDCA-2 (17598-08) in formulation buffer (10 mM Sodium Succinate, 150 mM L-Arginine HCl, 0.05% PS80, pH 6.0);

FIG. 8 is a non-limiting example of data generated from testing 150 mg/mL of DAC (17199-51-4) in formulation buffer (40 mM Succinate, 100 mM Sodium Chloride, pH 6.0);

FIG. 9 is a non-limiting example of data demonstrating the change of the open circuit potential measured by a Pt microelectrode in a solution of 10⁻²M Ru(NH₃)₆²⁺ in 0.1M KNO₃, 0.1M Na₂SO₄ and H₂O upon adding STX-100 (200 μg) and FB of the same volume as STX-100;

FIG. 10 is a non-limiting example of data demonstrating the effect of different concentrations and ratios of Ru(NH₃)₆³⁺/Ru(NH₃)₆²⁺ on potential measurements upon adding STX-100 (200 μg);

FIG. 11 is a non-limiting example of data demonstrating the average intra-day ΔE measurements for STX-100 and FB alone;

FIG. 12 is a non-limiting example of data demonstrating the ΔE due to various amounts of polysorbate 80; different volumes (2, 8, 12 μl) of polysorbate 80 were added to Redox solution;

FIG. 13 is a non-limiting example of data demonstrating the ΔE due to various amounts of arginine; different volumes (2, 4, 6 μl) of 150 mM arginine solution were added to Redox solution;

FIG. 14 is a non-limiting example of data demonstrating the ΔE when BART is in formulation buffer containing arginine and when BART is subjected to buffer exchange using the arginine free formulation buffer of STX-100; different volumes (3, 6, 9 μl) of BART with arginine or BART subjected to buffer exchange were added to Redox solution as indicated;

FIG. 15 is a non-limiting example of data demonstrating the ΔE when TWEAK is in formulation buffer containing arginine and when TWEAK is subjected to buffer exchange using the arginine free formulation buffer of STX-100; different volumes (3, 6, 9 μl) of TWEAK with arginine or TWEAK subjected to buffer exchange were added to Redox solution as indicated;

FIG. 16 is a non-limiting example of data demonstrating the ΔE when BDCA2 is in formulation buffer containing arginine and when BDCA2 is subjected to buffer exchange using the arginine free formulation buffer of STX-100; different volumes (3, 6, 9 μl) of BDCA2 with arginine or BDCA2 subjected to buffer exchange were added to Redox solution as indicated;

FIG. 17 is a non-limiting example of data demonstrating the effect of light exposure on ΔE measurements using STX-100 as an example;

FIG. 18 is a non-limiting example of data demonstrating the effect of temperature on ΔE measurements using STX-100 as an example; and

FIG. 19 is a non-limiting example of data demonstrating linear regression analysis for the oxidation analysis of STX-100 using the standard addition method as an example.

### DETAILED DESCRIPTION

Described herein are methods and systems for assessing pharmaceutical preparations. It should be appreciated that a pharmaceutical preparation can comprise one or more substances, including active as well as inactive substances. Active components of a preparation may include, for example, therapeutic proteins (e.g., antibodies), nucleic acids, small molecules, and others. Inactive components of a preparation, may include excipients, solubilizing agents, salts, buffers, and others. In some embodiments, a pharmaceutical preparation is a purified preparation of an active agent, such as, a therapeutic protein. In some embodiments, a pharmaceutical preparation is a purified preparation of an inactive agent, such as, an excipient (e.g., a polysorbate). In some embodiments, a pharmaceutical preparation is mixture one or more active agents and/or one or more inactive agents.

In some embodiments, methods and systems are provided for assessing oxidation of a pharmaceutical preparation. As used herein, the term “oxidation” generally refers to a loss of electrons from a chemical entity (e.g., molecule, atom or ion). In some embodiments, oxidation of proteins or other molecules may occur directly, for example, via a reaction with a reactive oxygen species. In some embodiments, oxidation of proteins or other molecules occurs indirectly, for example, via a reaction with by-products of oxidation. Various agents may bring about oxidation including agents, such as, H₂O₂ and HOCl, CCl₄, reduced transition metals such as Fe²⁺ or Cu⁺, γ-irradiation in the presence of O₂, ultraviolet light, oxidoreductase enzymes, by-products of lipid and free amino acid oxidation, and others.

With respect to proteins, it should be appreciated that any one or more amino acids may be oxidized. In some embodiments, amino acids prone to oxidation are cysteine and methionine, both of which contain susceptible sulfur atoms. In some embodiments, oxidizing species can induce modification of cysteine residues. For example, cysteine oxidation may lead to formation of disulfide bonds, mixed disulfides (e.g., with glutathione), and thiyl radicals. In some embodiments, oxidizing species can induce modification of methionine, e.g., forming methionine sulfoxide. Additional amino acids that are particularly prone to oxidation include histidine (His), phenylalanine (Phe), tryptophan (Trp), and tyrosine (Tyr) due to the high reactivity of their aromatic rings toward various reactive oxygen species.

As used herein the term “oxidation status” refers to the extent to which a chemical entity (e.g., molecule, atom or ion) is oxidized, e.g., the extent to which electrons have been lost from a molecule, atom or ion relative to a reference condition. In some embodiments, oxidation status is determined as an oxidation level (e.g., a relative oxidation level) of a chemical entity. In some embodiments oxidation status is determined as a percent oxidation of a chemical entity relative to a reference condition. In some embodiments, oxidation status is determined as a parameter (e.g., a binary parameter) indicative of whether or not a chemical entity possesses a threshold oxidation level, e.g., an oxidation level that is greater than a reference condition. Oxidation of proteins may be determined by assessing the extent to which individual amino acids have been oxidized relative to a reference condition. A reference condition may be the extent of oxidation of a protein in a formulation buffer or at a particular pH, for example. In some embodiments, percent oxidation of a reference standard is pre-determined from a focused peptide mapping analysis (e.g., using mass spectrometry or UV analysis for a particular protein under study). In some embodiments, the total percentage oxidation is determined as the sum of the oxidation of each peptide of a protein from the focused peptide mapping analysis, e.g., as determined by mass spectrometry. In some embodiments, susceptible sites for oxidation are determined using forced oxidized samples from peptide mapping analysis (e.g., as described for example, in Houde D, et al., Determination of protein oxidation by mass spectrometry and method transfer to quality control. J Chromatogr A. 2006 Aug. 11; 1123(2):189-98. Epub 2006 May 22, the contents of which are incorporated herein by reference).

It should be appreciated that a variety of oxidative modifications may occur in proteins. In some embodiments, oxidative modifications may involve disulfides, thiyl radicals, glutathiolation, methionine sulfoxide, carbonyls, 2-oxo-his dityrosine, chlorotyrosine, nitrotyrosine, tryptophanyl, hydroperoxides, lipid peroxidation adducts, amino acid adducts, glycoxidation adducts, cross-links, aggregates or fragments. As there are numerous types of protein oxidative modifications that can take place, there are also many methods for detecting and quantifying those modifications. Oxidation is traditionally measured using focused peptide mapping analysis (LC-MS), which is both costly and time consuming. However, oxidation measured using focused peptide mapping analysis or another method can be used to determine a standard-addition curve that enables one to relate potentiometric measurements to oxidation status (e.g., percent oxidation), as described herein.

In some embodiments, methods and systems are provided for assessing the oxidation status (e.g., percent oxidation) of a pharmaceutical preparation based on potentiometric techniques. In some embodiments, systems provided herein comprise at least an indicator electrode, a reference electrode, and a potential measuring device. In some embodiments, a redox potential is a potential difference between the electrodes in a reactive media (e.g., a solution). In some embodiments, a potential difference is related to one or more aspects of a component of the reactive medium, which may comprise one or more components of a pharmaceutical preparation (e.g., a therapeutic protein). In some embodiments, an indicator electrode is re-generated between measurements (e.g., by brief exposure to an acid (e.g., by dipping the electrode in 0.1M H₂SO₄ for 1-2 min, followed by rinsing with HPLC grade water)).

In some embodiments, an indicator electrode serves as an inert redox electrode (e.g., an electrochemically stable electrode), acting as a conductor by not giving up its own electrons to a corresponding reference electrode or solution. Any suitable indicator electrode may be used, including, for example, platinum, gold, palladium, or carbon based electrodes.

In some embodiments, a reference electrode provides a standard redox reaction that will accept or give up electrons to a solution. In some embodiments, a reference electrode acts as a half-cell with an accurately known electrode potential, E_{ref}, that is independent of the concentration of an analyte in solution. In some embodiments, a silver/silver chloride (Ag/AgCl) or a saturated calomel (Hg/Hg₂Cl₂) reference electrode may be used as a reference electrode.

As disclosed herein, indirect potentiometric methods provide an easy, fast and efficient assay to monitor oxidation (comparable to MS results, for example). Methods provided herein can be complete rapidly (e.g., within 15-20 minutes) and with high precision. In some embodiments, methods provided herein involve the use of indirect potentiometry, which is based on changes in electrode potential (ΔE) for a mediator system (redox couple) as a result of its interaction with oxidized species. Thus, in some embodiments, methods provided herein involve measuring a potential difference caused by a change in a solution upon completion of a reaction between a pharmaceutical preparation and a redox couple in the solution. As used herein the term “redox couple” refers to a combination of a reducing species and a corresponding oxidized species. In some embodiments, Ru(NH₃)₆³⁺/Ru(NH₃)₆²⁺ may be used as a redox couple. In some embodiments, hexacyanoferrate(III) and hexacyanoferrate(II) may be used as a redox couple. In some embodiments, iron(II) and iron(III) (ferrous/ferric) may be used as a redox couple.

In some embodiments the oxidation status (e.g., percent oxidation) of a pharmaceutical preparation (e.g., a therapeutic protein) is determined using potentiometric analysis. As a non-limiting example, when an oxidative species (Protein-Ox) is added to a redox couple containing solution e.g., Ru(NH₃)₆²⁺, the following reaction takes place:

Protein-Ox+Ru(NH₃)₆²⁺→Protein-Red+Ru(NH₃)₆³⁺ (1)

The potential of the redox electrode is governed by the concentration ratio of the redox couple and is expressed by the Nernst equation:

E₁=E°−(0.059/n)log([Ru(NH₃)₆²⁺]/[Ru(NH₃)₆³⁺]) (2)

Where E° is the formal redox potential of Ru(NH₃)₆³⁺/RuIII(NH₃)₆²⁺: E°=−0.173V vs Ag/AgCl; n=1. The redox electrode shows a Nernstian response in the Ru(NH₃)₆³⁺/Ru(NH₃)₆²⁺ solution down to 10⁻⁵ M.

When Protein-Ox is added and the reaction (1) is complete, the redox potential, E₂ equals:

E₂=E°−(0.059/n)log {([Ru(NH₃)₆²⁺]−[Protein-Ox])/([Ru(NH₃)₆³⁺]+[Protein-Ox])}; n=1 (3)

The change in potential of the redox electrode is governed by the change in the composition of the redox couple and can be expressed by:

ΔE=(0.059/n)log {(1+[Protein-Ox]/[Ru(NH₃)₆³⁺])/(1−[Protein-Ox]/[Ru(NH₃)₆²⁺)]}; n=1. (4)

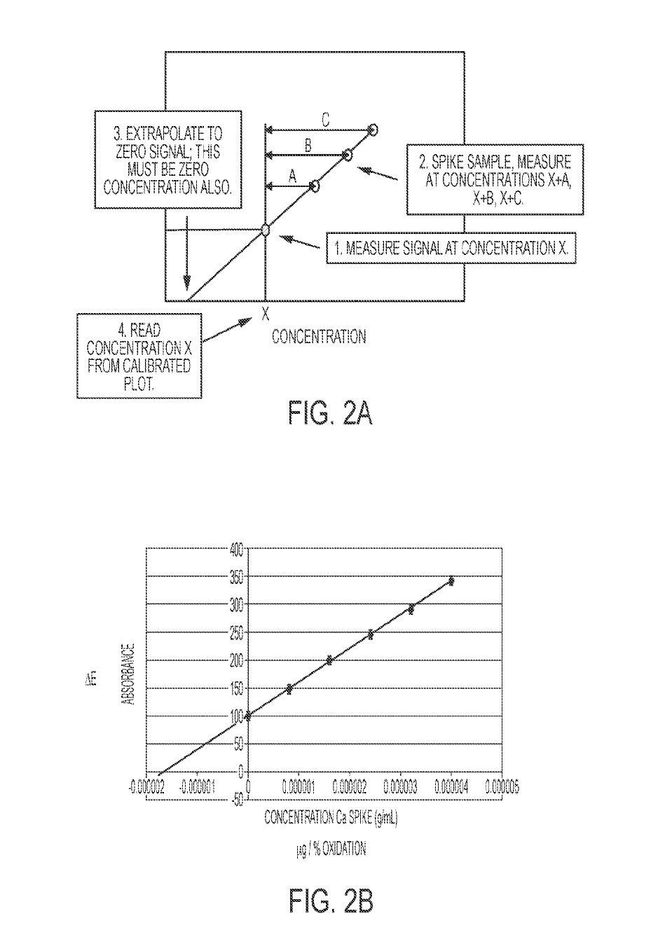

As shown in FIGS. 2A and 2B, the results may be plotted as:

{Change in potential (ΔE)} vs. {Total Amount of STD used/known % oxidation (μg/% oxidation)}

A regression line may be interpolated to the point on the x-axis at which y=0. This intercept on the x-axis (referred to as, X) corresponds to Total Amount of STD used divided by a known percentage oxidation (μg/% oxidation) in a test sample. This value is given by −a/b, the ratio of the intercept and the slope of the regression line. Therefore, since, X equals quantity (μg) divided by percent oxidation; it follows that % oxidation equals quantity (μg) divided by X. Thus, in some embodiments a potentiometric evaluation of a reference standard that relates a change in potential to an oxidation status (e.g., percent oxidation) or activity of the pharmaceutical preparation is made. In some embodiments, a potential difference (ΔE) may then be related to a standard where the level of oxidation (e.g., percent oxidation) or activity of the pharmaceutical preparation is known, as disclosed herein.

In some embodiments, an initial potential of a redox couple is corrected based on the change in potential caused by a formulation buffer (FB) of a pharmaceutical preparation (e.g., a therapeutic protein) or other preparation. As used herein the term, “formulation buffer (FB)” refers to a composition comprising one or more buffering agents. It should be appreciated that the degree of oxidation for a given component of a pharmaceutical preparation may affect the ratio of the reducing species and its oxidized form e.g., Ru²⁺:Ru³⁺, which is measured by potentiometry (indirect potentiometry).

In some embodiments, a FB for a pharmaceutical preparation (e.g., a composition containing a therapeutic protein) may contain a variety of excipients to stabilize proteins, act as antimicrobials, aid in the manufacture of the dosage form, control or target drug delivery and minimize pain upon injection. In some embodiments, a formulation buffer does not contain a polysorbate or an amino acid (e.g., arginine). Notably, in some embodiments, these excipients may affect the redox couple and it may therefore be important to determine the potential difference (ΔE) caused by the FB in order to account for and/or eliminate matrix effects and to determine the potential difference caused by the therapeutic protein of unknown oxidation level. In some embodiments, the therapeutic protein sample of unknown oxidation level is then added and potential change is measured. In some embodiments, quantitation is performed using the standard addition method. In some embodiments, a solution is further spiked with samples of known % oxidation (measured by MS) at different amounts (two STD additions) followed by potentiometric measurements.

In some embodiments, a therapeutic protein evaluated according to methods disclosed herein is an antibody. In some embodiments, the antibody is STX-100, Tysabri, Daclizumab (DAC), BART, Tweak, or Anti-BDCA2. STX-100 is a humanized monoclonal antibody that targets integrin αvβ6. STX-100 exhibits significant anti-fibrotic activity in preclinical animal models of kidney, lung and liver disease. The FDA has previously granted orphan drug designation to STX-100 for chronic allograft nephropathy. TYSABRI (Natalizumab) is a humanized monoclonal antibody against the cell adhesion molecule α4-integrin. Natalizumab is used in the treatment of multiple sclerosis and Crohn's disease. BART (BIIB037) is an anti-beta-amyloid human monoclonal antibody used as a treatment for Alzheimer's disease (AD). It is believed that BIIB037 binds to and eliminates toxic amyloid plaques that form in the brains of patients with AD, thereby potentially suppressing the progression of the disease. Anti-TWEAK is a humanized monoclonal antibody specific for TWEAK useful in the treatment of lupus nephritis (LN). Daclizumab (Zenapax) is a therapeutic humanized monoclonal antibody used to prevent rejection in organ transplantation, especially in kidney transplants. Daclizumab works by binding to CD25, the alpha subunit of the IL-2 receptor of T cells.

In some embodiments, an antibody evaluated according to methods disclosed herein is selected from: anti-LINGO, anti-LINGO-1, interferon (e.g., interferon beta 1a—AVONEX), Abciximab (REOPRO®), Adalimumab (HUMIRA®), Alemtuzumab (CAMPATH®), Basiliximab (SIMULECT®), Bevacizumab (AVASTIN®), Cetuximab (ERBITUX®), Certolizumab pegol (CIMZIA®), Daclizumab (ZENAPAX®), Eculizumab (SOLIRIS®), Efalizumab (RAPTIVA®), Gemtuzumab (MYLOTARG®), Ibritumomab tiuxetan (ZEVALIN®), Infliximab (REMICADE®), Muromonab-CD3 (ORTHOCLONE OKT3®), Natalizumab (TYSABRI®), Omalizumab (XOLAIR®), Palivizumab (SYNAGIS®), Panitumumab (VECTIBIX®), Ranibizumab (LUCENTIS®), Rituximab (RITUXAN®), Tositumomab (BEXXAR®), and Trastuzumab (HERCEPTIN®). In some embodiments, the antibody is Natalizumab (TYSABRI®).

In some embodiments, an antibody evaluated according to methods disclosed herein is selected from Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab, Tildrakizumab, Tigatuzumab, TNX-, Tocilizumab, Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

It should be appreciated that while methods disclosed herein are useful for evaluating immunoglobulins and fragments thereof, any protein or biomolecule may be suitable evaluated using methods provided herein. In some embodiments, a protein of interest is a blood cascade protein. Blood cascade proteins are known in the art and include, but are not limited to, Factor VII, tissue factor, Factor IX, Factor X, Factor XI, Factor XII, Tissue factor pathway inhibitor, Factor V, prothrombin, thrombin, vonWillebrand Factor, kininigen, prekallikrien, kallikrein, fribronogen, fibrin, protein C, thrombomodulin, and antithrombin. In some embodiments, the blood cascade protein is Factor IX or Factor VIII. It should be appreciated that methods provided herein are also applicable for uses involving the production of versions of blood cascade proteins, including blood cascade proteins that are covalently bound to antibodies or antibody fragments, such as Fc. In some embodiments, the blood cascade protein is Factor IX-Fc (FIXFc) or Factor VIII—Fc (FVIIIFc). In some embodiments, one or more proteins of interest are hormones, regulatory proteins and/or neurotrophic factors. Neurotrophic factors are known in the art and include nerve growth factor (NGF), brain-derived neurotrophic factor (BDNF), neurotrophin-3 (NT-3), neurotrophin-4 (NT-4), members of the glial cell line-derived neurotrophic factor ligands (GDNF) and ciliary neurotrophic factor (CNTF). In some embodiments, the protein of interest is neublastin.

### Systems for Potentiometric Evaluation

Aspects of the disclosure provide systems for assessing pharmaceutical preparations potentiometrically. In some embodiments, systems are useful for determining the oxidation status of pharmaceutical preparations. Systems provided herein typically comprise at least one container configured for housing a solution to be analyzed potentiometrically. In some embodiments, systems are configured with an indicator electrode and/or a reference electrode. In some embodiments, systems are configured with a micro-indicator and a micro-reference electrodes (alternatively a micro combination redox electrode is used). In some embodiments, an indicator electrode and reference electrode are disposed in a container. Systems provided herein also typically comprises a potential measuring device operably connectable to the indicator electrode and reference electrode and configured for obtaining one or more potential measurements between an indicator electrode (e.g., micro-indicator electrode) and reference electrode (e.g., micro-reference electrode).

For multi-throughput implementations, systems may comprise a plurality of containers (e.g., wells of a multi-well plate, e.g., a 96 well plate). In such implementations, indicator electrodes and/or reference electrodes (or micro combination redox electrodes) may be provided for each of a plurality of containers. For example, a multi-throughout implementation may comprises a multi-well plate (e.g., a 96-well plate, 384-well plate) fitted with a pair of indicator and reference electrodes (or micro combination redox electrodes) for each well of plate. An electrode assembly may be provided that can be fitted with a multi-well plate format for purposes of disposing pairs of electrodes (or micro combination redox electrodes) or individual electrodes into wells of the plate. An electrode assembly may comprise electrodes pairs (or micro combination redox electrodes) or individual electrodes for each well. For example, an electrode assembly for a 96-well plate, may comprise 96 pairs of electrodes (or micro combination redox electrodes) that are configured (e.g., in 8 rows of 12) to be disposable (e.g., simultaneously) in each well of the plate. For example, electrodes can be aligned in arrays in an electrode assembly manifold such that the placement of the individual electrodes or electrode pairs corresponds to the placement of the wells in the multi-well plate. In some embodiments, a portion of a well or container (e.g., a container bottom) may serve as an electrode.

However, in some embodiments, an electrode assembly may have fewer electrode pairs than wells. For example, an electrode assembly for a 96-well plate, may comprise 8 pairs of electrodes that are disposable (e.g., simultaneously) in single column of wells of the plate. Similarly, an electrode assembly for a 96-well plate, may comprise 12 pairs of electrodes that are disposable (e.g., simultaneously) in single row of wells of the plate. A system may be fitted with a robotic system to control the positioning of electrodes into and out from a container or containers.

Systems provided herein may be fitted with a computer operably connectable to a potential measure device and configured to receive and process information from the potential measuring device. In some embodiments, a system may be fitted with a robotic system to control the positioning of electrodes into and out from a container or containers, and the computer may be configured to control operation of the robotics system. In some embodiments, a computer may be configured for determining the oxidation status (e.g., percent oxidation) of a pharmaceutical preparation based on one or more potential measurements obtained from the potential measuring device while the pharmaceutical preparation is present in the solution. A computer may be configured for determining the oxidation status (e.g., percent oxidation) of the pharmaceutical preparation based on a change in potential between an indicator electrode and a reference electrode resulting from presence of the pharmaceutical preparation in the solution that comprises a redox couple. A computer may also be configured to determine the oxidation status (e.g., percent oxidation) of a protein based on a reference standard that relates potential differences to a mass spectrometrically determined oxidation status (e.g., percent oxidation) of the pharmaceutical preparation.

A computer may comprise an input interface configured to receive information from a potential measuring device indicative of one or more potential differences measured between an indicator electrode and reference electrode; and/or at least one processor programmed to evaluate a model that relates the one or more potential differences to the oxidation status (e.g., percent oxidation) of the pharmaceutical preparation. A computer may also comprise an output interface configured to output a signal indicative of the oxidation status (e.g., percent oxidation).

### Computer Implementations

It should be appreciated that methods disclosed herein may be implemented in any of numerous ways. For example, certain embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a smart phone, tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools (e.g., MATLAB), and also may be compiled as executable machine language code or intermediate code that is executed on a framework or virtual machine.

In this respect, aspects of the disclosure may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with information (e.g., potentiometric measurement information) and/or one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above. As used herein, the term “non-transitory computer-readable storage medium” encompasses only a computer-readable medium that can be considered to be a manufacture (e.g., article of manufacture) or a machine.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

As used herein, the term “database” generally refers to a collection of data arranged for ease and speed of search and retrieval. Further, a database typically comprises logical and physical data structures. Those skilled in the art will recognize methods described herein may be used with any type of database including a relational database, an object-relational database and an XML-based database, where XML stands for “eXtensible-Markup-Language”. For example, potentiometric measurement information may be stored in and retrieved from a database. The potentiometric information may be stored in or indexed in a manner that relates potentiometric measurements with oxidation status (e.g., percent oxidation) or activity levels, or with a variety of other relevant information.

Computer-executable instructions may be in many forms, such as program modules, executed by one or more computers or other devices. Generally, program modules include routines, programs, objects, components, data structures, etc. that perform particular tasks (e.g., tasks relating to oxidation status (e.g., percent oxidation) determinations) or implement particular abstract data types. Typically the functionality of the program modules may be combined or distributed as desired in various embodiments.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1: Technical Development Method Protocol

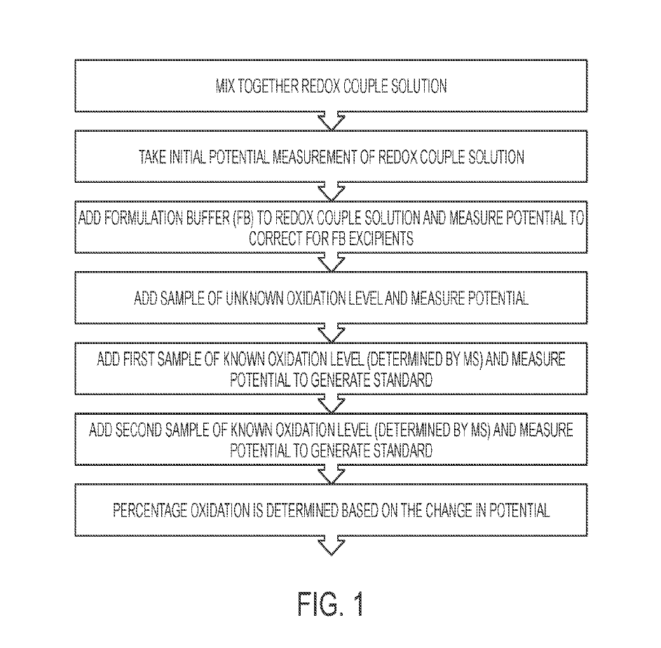

Provided herein is a method to assess the oxidation level of different proteins using an indirect potentiometric analysis. A flow diagram of the method for determining the oxidation of proteins using indirect potentiometric analysis is provided in FIG. 1. This method pertains to the analysis of different proteins using an indirect potentiometric analysis to determine the percentage oxidation. The method involves a 2 point standard addition. The percentage oxidation of the standard is known from pre-determined results obtained from focused peptide mapping analysis by mass spectrometry and using this as a basis for further calculations, the unknown percentage oxidation of the samples under analysis can be determined quickly and easily by plotting an X-Y scatter graph and performing simple mathematical calculation. In this example, ΔE is the change in potential after addition of the sample or standard with respect to the corrected potential after formulation buffer (FB) addition.

### Solution Preparation:

To prepare a redox couple, a Hexaamineruthenium (II) chloride stock solution of 0.1M, 27.30 mg of Hexaamineruthenium (II) chloride was added to 1 mL of HPLC grade water and used within 1 hour following preparation to minimize oxidation of the stock.

A Hexaamineruthenium (III) chloride stock solution of 0.001M was made by adding 30.91 mg of Hexaamineruthenium (III) chloride to 1 mL of HPLC grade water. From this solution, 100 μL was added to 900 μL of HPLC grade water to result in 0.01M Ru³⁺. From the 0.01M Ru³⁺ solution, 100 μL was added to 900 μL of HPLC grade water to result in 0.001M Ru³⁺. This solution was used within 1 hour of preparation. A working redox solution (10-2M Ru2+/10-4M Ru3+ in HPLC grade water) was made by adding 15 μL of 0.1M Ru²⁺ and 15 μL of 0.001M Ru³⁺ to 120 μL of HPLC grade water to result in 150 μL of 10⁻²M Ru²⁺/10⁻⁴ M Ru³⁺. The solution was vortexed gently for a few seconds such that the redox solution was mixed completely. Materials (Table 1) and equipment (Table 2) used in this example are outlined below:

TABLE 1
Materials

| Material Name | Supplier | Part Number |
|---|---|---|
| Hexaamineruthenium(III) Chloride | Sigma-Aldrich | Part number: 262005 |
| Hexaamineruthenium(II) Chloride | Sigma-Aldrich | Part number: 303690 |
| Sulfuric acid (2-5M) | Across | Part number: |
| Water, HPLC grade | Baker | Part number: 4218- |
| | | 03, or equivalent |
| 10 mM Sodium Citrate, 5% (w/v) | Biogen Idec | Media Prep |
| Sucrose, pH 6.1 (FB_STX-100) | | |
| Formulation buffer of different | Biogen Idec | Media Prep |
| proteins under study | | |

TABLE 2
Equipment

| Equipment Name | Supplier | Model Number |
|---|---|---|
| pH 700 Series Voltmeter | Oakton | Serial number: 2023401 |
| Centrifuge | Sorvall | Part number: RT7, or |
| | | equivalent |
| Amicon centrifugal devices (MWCO 10,000) | Millipore | Part number: UFC801096 |
| Micro-ORP Electrode/MI-800 Series | Microelectrode, | 407E; BNC#93372 |
| | Inc. | |
| Micro-reference Electrode with Glass | Microelectrode, | MI-401F Pin#92913 |
| Barrel and Frit Junction, Pin#92913 | Inc. | |
| Nanodrop 1000 | Thermo Fisher | |

### Standard Curve and Assay Control Preparation:

Following the addition of formulation buffer (FB), unknown sample and the two reference standards (with pre-determined % oxidation) to the redox solution, a change in potential (ΔE) was measured. An indicator electrode is regenerated between the measurements by dipping it into 0.1M H₂SO₄ solution for 1-2 min, followed by rinsing with HPLC grade water.

To calculate the % oxidation of the unknown sample a curve was plotted using (1) the change in potential (ΔE) vs. (2) the total amount of protein used/known % oxidation (μg/% oxidation). In this example, calculation of the unknown value of X was performed using a linear fit and interpolating the graph. Since, X=μg/% oxidation, the % oxidation may be determined by the following relation:

% oxidation=μg/X.

The accuracy in this case can be determined by making comparisons to reference standards having known oxidation level (e.g., percent oxidation).

For assay controls, percent oxidation was determined for a particular protein under study. For example, the total percentage oxidation of a particular protein was determined as the sum of the % oxidation of each peptide of that protein that was evaluated by focused peptide mapping analysis. This oxidation value was used as the basis to perform further calculations.

### Procedure:

Potentiometric analysis was performed using the Hexaamineruthenium (II/III) chloride redox solution. Potentials were measured by inserting the reference and microelectrode in the vial containing the solution. To measure the initial potential, 150 μL of freshly prepared Hexaamineruthenium (II/III) chloride redox solution in a 0.7 mL vial was measured. Next, 1.5 μL of formulation buffer (FB) (for example, the FB used with STX-100) was added and the potential was measured to correct the initial potential of the redox solution for any change in potential due to excipients. Notably, the amount of FB added for correction may differ among proteins due to different concentrations of the proteins under study. The following steps were conducted: (1) 100 μg of unknown sample was added and the potential was measured, (2) 100 μg of STD1 was added and the potential was measured, (3) 100 μg of STD 2 was added and the potential was measured. An indicator electrode was re-generated between the measurements by dipping it for 1-2 min into 0.1M H₂SO₄ solution followed by rinsing with HPLC grade water.

Buffer Exchange was used for samples containing arginine in a formulation buffer. For example, 100 μL of either unknown samples, assay controls or blanks were pipetted into a labeled filter tubes. Next, 3900 μL of formulation buffer (STX-100) (used for buffer exchange for all the proteins with Arginine in FB) was added to reach final volume of 4000 μL. Samples were centrifuged at 3800±200 rpm at 5° C. until sample volumes were reduced to 100 μL. In this example, it took approximately 17 minutes to reduce the volume from 4 mL to 100 μL using a Sorvall, RT17 centrifuge. A second wash was performed to further remove Arginine. Samples were centrifuged at 3800±200 rpm at 5° C. until sample volumes are reduced to 100 μL. Concentration of the buffer exchanged protein was measured using a Nanodrop 1000. Notably, the correct extinction coefficient must be used for accurate concentration measurements. The found concentration was used to calculate volumes required for 100 μg protein.

### Data Analysis and Processing:

All potential measurements observed were evaluated to calculate the change in potential with respect to each sample/STD addition. A curve was plotted between: {Change in potential (ΔE)} vs. {Total Amount of protein used/known % oxidation (μg/% oxidation)}. An “XY Scatter” plot of the peak area versus theoretical amount was created. A linear trend line of the data was created; set the y-intercept to 0; displayed R² and the line equation on the graph. In this example, the minimal acceptable R² value was 0.98. The unknown value of X was calculated using a linear fit and interpolating the graph. Since, X=μg/% oxidation, the Oxidation=μg/X. The accuracy was determined by comparing with known % oxidation of the reference standard.

### Example 2: Optimizing Parameters

### Effect of Ionic Strength:

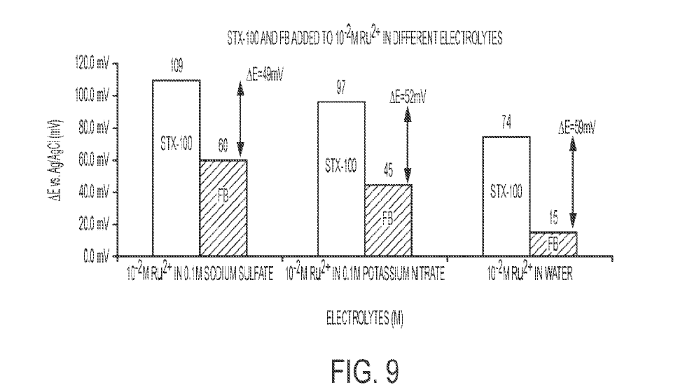

The ionic strength of a solution is a measure of the concentration of ions in that solution. It was found that precipitation of the protein occurs in 1M Na₂SO₄, 1M Sodium Citrate or 1M KNO₃. Potential measurements using 10 mM, 50 mM and 100 mM electrolytes resulted in unstable potentials. Notably, a potential change due to formulation buffer (FB) addition was significantly higher for different electrolytes as compared to water (FIG. 9). In this example, STX-100 and FB were added to 10⁻²M Ru²⁺ in water and in 0.1M sodium sulfate and potassium nitrate solutions. The potential change (ΔE) between FB and STX-100 in water was 59 mV while the potential change in sodium sulfate and potassium nitrate were 49 mV and 52 mV respectively.

### Optimizing the Redox Couple Solution:

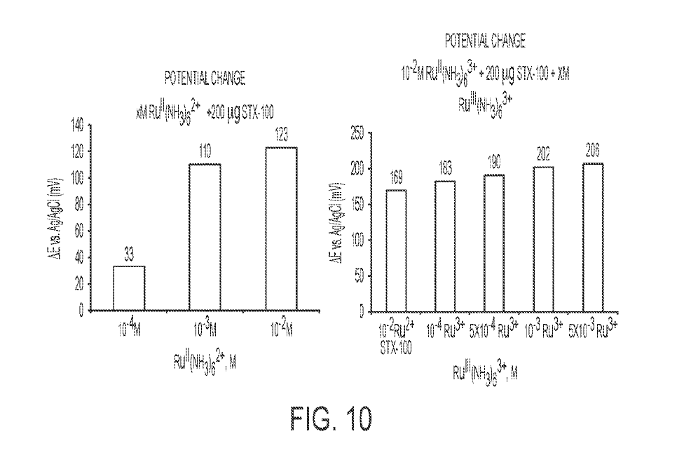

Optimization of the Ru(NH₃)₆³⁺/Ru(NH₃)₆²⁺ redox couple ratio is necessary to assess the maximum potential change due to interaction with the oxidized protein. Using the protein STX-100 as an example, the following formula summarizes the redox reaction between the redox couple solution and the therapeutic protein STX-100: STX-100-Ox+Ru(NH₃)₆²⁺→STX-100-Red+Ru(NH₃)₆³⁺. The data revealed that a maximum and stable potential change was observed using 10⁻²/10⁻⁴M of Ru(NH₃)₆³⁺/Ru(NH₃)₆²⁺ redox couple solution in water (FIG. 10).

### Intra-Day Precision:

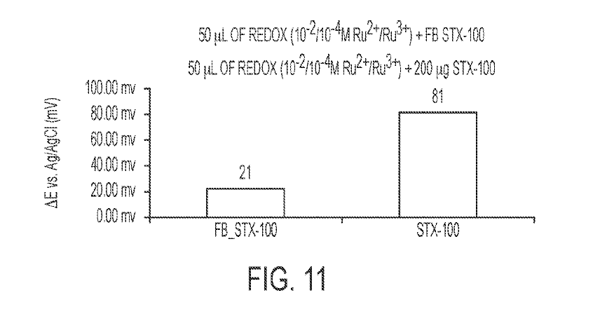

To test intra-day precision, using STX-100 as an example, seven ΔE (mV) measurements for STX-100 in FB (STX-100) and six ΔE (mV) measurements for formulation buffer alone (FB_STX-100) were made within a single day using the redox couple solution 10⁻²/10⁻⁴M of Ru(NH₃)₆³⁺/Ru(NH₃)₆²⁺ (Table 3 and FIG. 11). The formulation buffer used for STX-100 was 10 mM Sodium Citrate 5% (w/v) sucrose, pH 6.1. The average ΔE for STX-100 was 81 mV with an RSD of 7.5% and the average ΔE for FB_STX-100 was 21 mV with an RSD of 12.9%.

TABLE 3
Intra-day measurements of ΔE for STX-100 and FB.

| | | DE |
|---|---|---|
| Sample | Volume | (mV) |
| 10⁻²/10⁻⁴ | 50 mL | NA |
| Ru(NH₃)₆³⁺/Ru(NH₃)₆²⁺ | | |
| in H₂O | | |
| STX-100 | 3 μL (200 μg) | 83 |
| STX-100 | 3 μL (200 μg) | 90 |
| STX-100 | 3 μL (200 μg) | 80 |
| STX-100 | 3 μL (200 μg) | 82 |
| STX-100 | 3 μL (200 μg) | 77 |
| STX-100 | 3 μL (200 μg) | 83 |
| STX-100 | 3 μL (200 μg) | 71 |
| Average ΔE_STX-100 | 3 μL (200 μg) | 81 mV |
| Standard Deviation | n = 7 | 6.1 |
| % RSD | n = 7 | 7.5 |
| Average ΔE_FB_STX- | | |
| 100 | 3 μL | 21 mV |
| Standard Deviation | n = 6 | 2.5 |
| % RSD | n = 6 | 12.9 |

### Potential Change (ΔE) Due to Polysorbate 80 and Arginine:

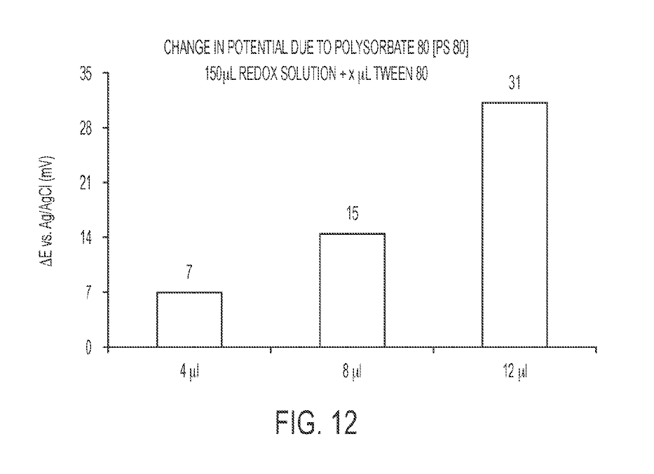

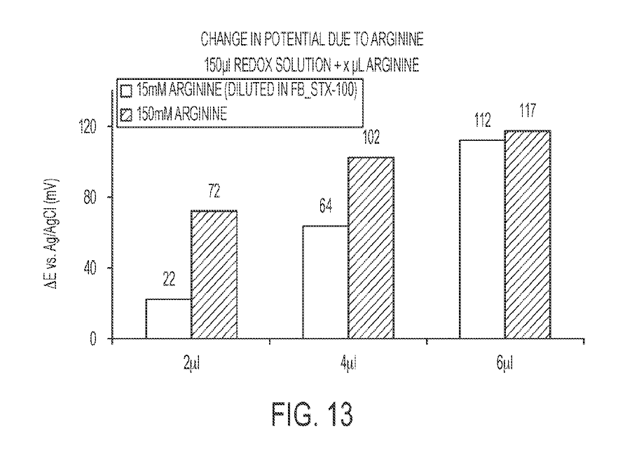

When using potentiometry to measure the oxidation level of a therapeutic or component in a buffer or pharmaceutical preparation, it is advantageous in some cases to determine if, and to what degree, any excipients affect the potential (ΔE) measurements in order to accurately determine the percent oxidation of a given therapeutic or component. In this example, the effect of polysorbate 80 (FIG. 12) and arginine (FIG. 13) on ΔE measurements was determined. It was found that polysorbate 80 does not affect the potential as much as arginine. Therefore, dilution or buffer exchange may not be necessary when formulation buffers contain polysorbate 80. Alternatively, arginine had a significant effect on potential of the redox solution with unstable readings. Diluting the formulation buffer 1:10 or 1:20 did reduce the large change in potential. Notably, the dilution here is followed by a buffer exchange using the STX-100 formulation buffer.

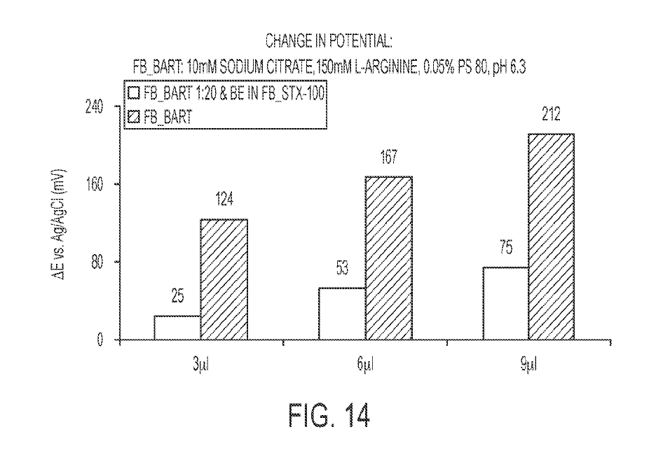

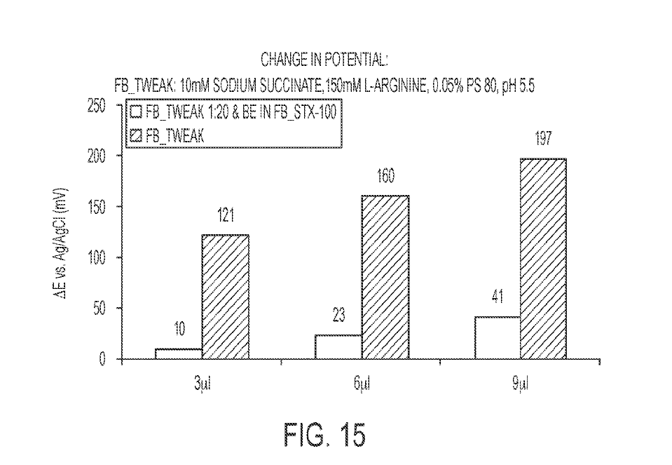

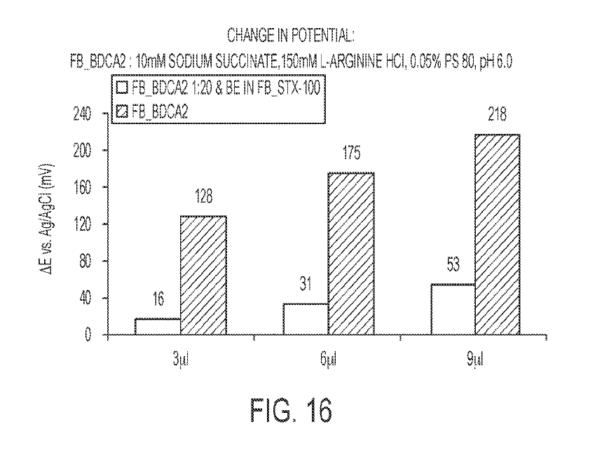

Buffer exchange can be used to remove excipients, such as arginine, from a composition that affect the potential of a redox couple. This allows for a more accurate measurement of percent oxidation of a given therapeutic or component in a buffer or pharmaceutical preparation. For example, when the therapeutic proteins BART, TWEAK, and BDCA2 were in formulation buffer containing arginine, the ΔE measurements were significantly higher when compared to the ΔE measurements following buffer exchange to the STX-100 formulation buffer, which does not contain arginine (FIGS. 14-16). Thus, buffer exchange in some instances allows for the accurate determination of potential, and therefore oxidation level, of a component within a pharmaceutical preparation or buffer.

In one example, change of open circuit potential was measured by a Pt microelectrode in 150 μL of 10⁻²M/10⁻⁴ M Ru(NH₃)₆²⁺/Ru(NH₃)₆³⁺ upon adding FB_Tweak before and after 1:20 dilution with HPLC grade H₂O and buffer exchange into 10 mM Sodium Citrate, 5% (w/v) sucrose, pH 6.1. Notably, interference from arginine was substantially less when buffer exchange into 10 mM Sodium Citrate, 5% (w/v) sucrose, pH 6.1 was used after the dilution step. In some embodiments, this allowed for more accurate measurement of percent oxidation of therapeutic protein.

### Stability of the Redox Couple Solution:

The redox couple solution (150 μL 10⁻² M/10⁻⁴ M of Ru(NH₃)₆³⁺/Ru(NH₃)₆²⁺) was made by diluting Hexamamineruthenium(II)chloride and Hexamamineruthenium(III)chloride in HPLC grade water. To determine the stability of the redox couple solution, E vs. Ag/AgCl (mV) was measured at 0 min, 5 min, 10 min, and 15 min after making the redox couple solution to determine ΔE over time (Table 4). It was found that the redox solution was stable up to 15 minutes (having a ΔE less than 20 mV), which was sufficient for all potential measurements made.

TABLE 4
Change in the potential (ΔE) of the
redox couple solution over time.

| Time | E vs. Ag/AgCl (mV) | ΔE (mV) |
|---|---|---|
| 0 | −59.3 | NA |
| 5 min | −58.2 | 1.1 |
| 10 min | −54.9 | 4.4 |
| 15 min | −50.6 | 8.7 |

### Method of Standard Addition:

To determine the % oxidation of a component within a buffer or pharmaceutical preparation, for example a therapeutic protein, using indirect potentiometry the potential of the buffer or pharmaceutical preparation having a component with a known % oxidation level may be used. For example, the change in potential (ΔE) of the redox couple solution can be measured after the addition of formulation buffer, the addition of a sample in formulation buffer having an unknown oxidation level, and the addition of a sample (reference standard) in formulation buffer having a known % oxidation level. The reference standard is added at two different amounts to plot the data. A curve can be plotted between: {change in potential (ΔE)} vs. {total amount of protein used/known % oxidation (μg/% oxidation)}. The unknown value of X can be calculated using a linear fit and interpolating the graph. Since, X=μg/% oxidation, the % oxidation=μg/X. The accuracy can be determined by comparing with the known % oxidation of the reference standard. The indicator electrode is regenerated each time the solution is changed by dipping it into 0.1M H₂SO₄ for 1-2 min followed by HPLC water rinse.

### Intra-Day Precision:

Intra-day precision was assessed by performing potentiometric measurements with STX-100 as an example. Six measurements of potential change for STX-100 and six measurements of potential change for formulation buffer alone have been performed using the redox couple solution 10-2/10-4M of Ru(NH3)63+/Ru(NH3)62+ within a single day (Table 5). The formulation buffer of STX-100 is 10 mM Sodium Citrate 5% (w/v) sucrose, pH 6.1. A fresh redox solution was prepared every time before addition of FB and STX-100 to facilitate a reproducible zero point measurement. The average data for potential change for STX-100 was found to be 81 mV with an RSD of 7.5% and the average potential change, ΔE for FB was 21 mV with an RSD of 12.9% (Table 5).

TABLE 5
Repeatability data for potential changes during the reaction
of A- FB (3 μl) and B- STX-100 (3 μl of 65 mg/mL) with 10⁻²/10⁻⁴ M
of Ru(NH₃)₆³⁺/Ru(NH₃)₆²⁺ (50 μL)

| Chemical | | | | | | | | | |
|---|---|---|---|---|---|---|---|---|---|
| Replicates | S1 | S2 | S3 | S4 | S5 | S6 | AVERAGE | STDEV | % RSD |
| ΔE, mV | 20 | 25 | 18 | 21 | 18 | 21 | 21 | 2.6 | 12.6 |
| ΔE, mV | 83 | 90 | 80 | 82 | 77 | 83 | 83 | 4.3 | 5.2 |

### Intermediate Precision:

Method intermediate precision was assessed by performing potentiometric measurements of six chemical replicates of STX-100 and FB on three different days, using the procedure described above. The precision data are summarized in Table 6.

The calculated values of intermediate precision for potentiometric measurements were determined to be 21 mV with an RSD of 11.7% for FB and 87 mV with an RSD of 5.0% for STX-100 (Table 6). The results indicate good precision for monitoring potential changes as a result of FB and protein interactions with a mediator using this assay.

TABLE 6
Intermediate Precision Data for Potential Changes during the
Reaction of A- FB (3 μL) and B- STX-100 (3 μL of 65 mg/mL)
with 10⁻²/10⁻⁴ M of Ru(NH₃)₆³⁺/Ru(NH₃)₆²⁺ (50 μL)

| | Day 1 | Day 2 | Day 3 | | | |
|---|---|---|---|---|---|---|
| | (n = 6) | (n = 6) | (n = 6) | AVERAGE | STDEV | % RSD |
| ΔE, mV | 21 | 24 | 18 | 21 | 2.4 | 11.7 |
| ΔE, mV | 89 | 82 | 90 | 87 | 4.4 | 5.0 |

### Example 3: Using Indirect Potentiometry to Determine % Oxidation of Pharmaceutical Preparations

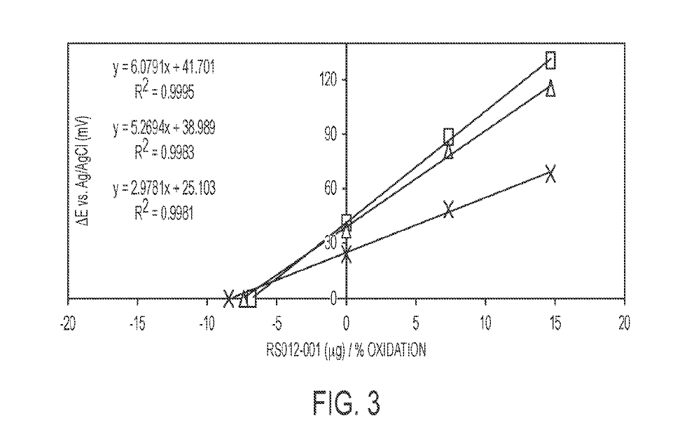

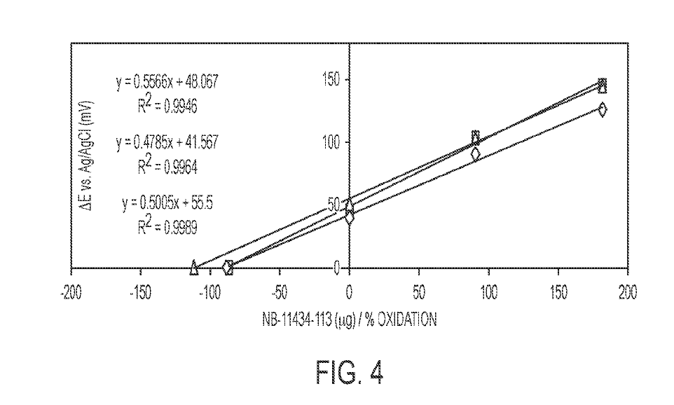

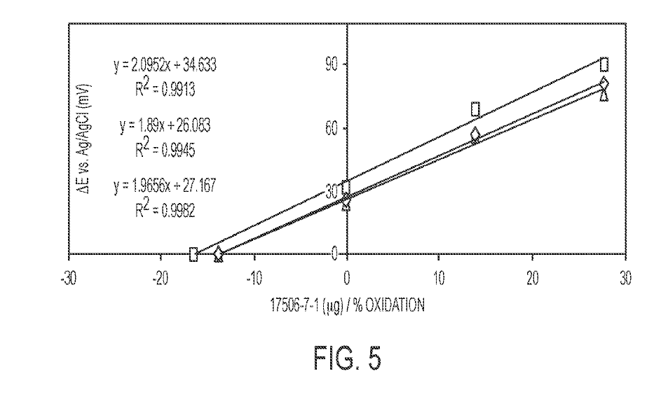

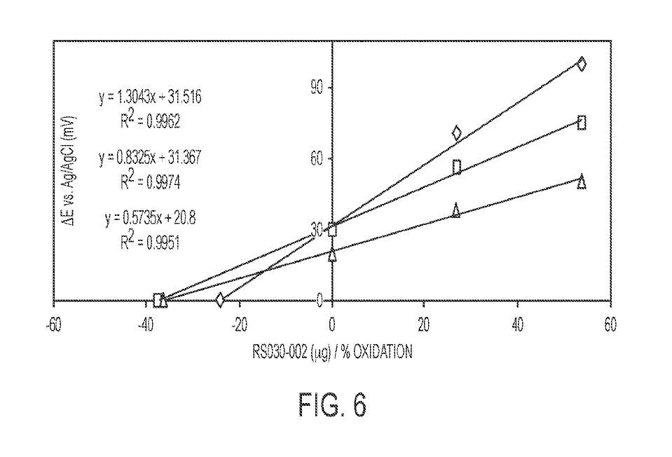

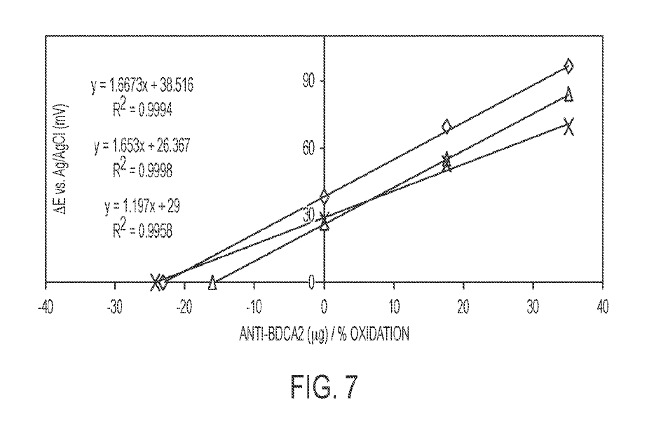

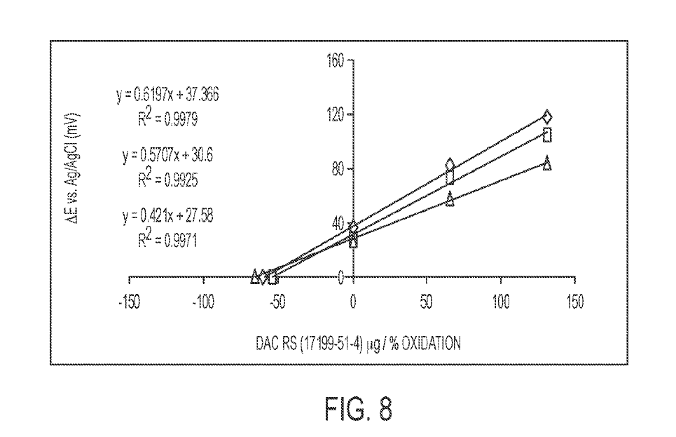

The % oxidation of a panel of therapeutic proteins (Table 7) was determined using indirect potentiometry where the percent oxidation of the reference standards was pre-determined by mass spectrometry. The redox couple used for each experiment was 10⁻²M/10⁻⁴ M of Ru(NH₃)₆³⁺/Ru(NH3)₆²⁺. Each experiment was performed in triplicate to determine the average % oxidation of the sample and the % relative standard deviation (RSD). Accuracy was determined by comparing with the known % oxidation of the reference standard. The therapeutic proteins STX-100 (FIG. 3 and Table 8), Tysabri (FIG. 4 and Table 9) and Daclizumab (DAC) (FIG. 8 and Table 13) were measured in their respective formulation buffers (Table 7) as they do not contain arginine. The therapeutic proteins BART (FIG. 5 and Table 8), Tweak (FIG. 6 and Table 9), and Anti-BDCA2 (FIG. 7 and Table 12) were diluted 1:20 followed by buffer exchange as their formulation buffers contained arginine (e.g., 140 mM arginine). The calculated values of intra-assay precisions for % oxidation using indirect potentiometric measurements were determined to be 9.7%-24.8% RSD for the six proteins with the % oxidation ranging between 1.1% and 13.3%. The % oxidation determined for each sample was within ±30% of the expected % oxidation determined by mass spectrometry.

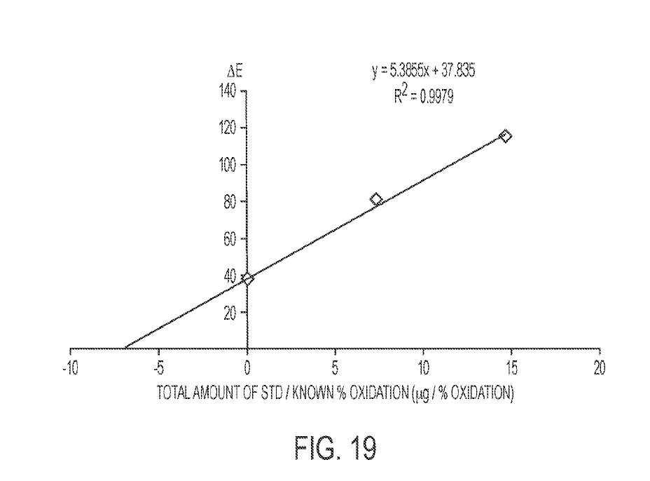

Each experiment was performed in triplicate to determine the average % oxidation of sample and the % relative standard deviation (RSD). The linear regression analysis was applied to derive the % oxidation of studied protein as described above. FIG. 19 demonstrates standard addition method used to determine STX-100 oxidation. The data is accompanied by good linearity observed upon addition of protein and two STDs to the redox couple (FIG. 19). Accuracy determined by comparing with the known % oxidation of the reference standard showed that % oxidation determined for each sample was within ±20% of the expected % oxidation determined by mass spectrometry. The data indicates that the method is suitable to monitor protein oxidation ranging between 1.1% and 13.6% (Table 14).

TABLE 7
Therapeutic proteins used to test the accuracy of indirect potentiometry
in predicting % oxidation level, including the protein identity,
the protein concentration and the formulation buffer used.

| Protein | Concentration | | Formulation Buffer |
|---|---|---|---|
| STX-100 | 65 | mg/mL | 10 mM Sodium Citrate 5% |
| (RS012-001) | | | (w/v) sucrose, pH 6.1 |
| Tysabri | 25 | mg/mL | 10 mM Sodium Phosphate, |
| (NB-11434-113) | | | 140 mM NaCl, pH 6.1 |
| BART | 51.7 | mg/mL | 10 mM Sodium Citrate, 150 mM |
| (17506-7-1) | | | L-Arginine, 0.05% PS 80, pH 6.3 |
| Tweak | 100 | mg/mL | 10 mM Sodium Succinate, |
| (RS030-002) | | | 150 mM L-Arginine, pH 5.5 |
| Anti-BDCA-2 | 48.5 | mg/mL | 10 mM Sodium Succinate, 150 mM |
| (17598-08) | | | L-Arginine HCl, 0.05% PS80, pH 6.0 |
| DAC | 150 | mg/mL | 40 mM Succinate, 100 mM Sodium |
| (17199-51-4) | | | Chloride, pH 6.0 |

TABLE 8
Results of testing STX-100 formulated in 10 mM Sodium Citrate, 5%(w/v) sucrose, pH 6.1.

| | | | | | | | | | Found % | | | |
|---|---|---|---|---|---|---|---|---|---|---|---|---|
| | | | | STX-100 | | | Un- | | Oxidation = | | Average % | |
| | | | | (μg)/% | | | known = | Expected % | STX-100(ug)/ | Accu- | Oxidation | % |
| No. | Sample | Δ ul | Δ ug | Oxidation | E | ΔE | X | Oxidation | X | racy | (n = 3) | RSD |
| 1 | Redox | 150 ul | | Zero Point | −34.2 | 0 | | | | | 13.3% | 10.2 |
| | unknown | 1.5 ul | 100 | 0 | 6.6 | 40.8 | 6.86 | 13.6 | 14.6 | 107.2% | | |
| | STD1_RS012-001 | 1.5 ul | 100 | 7.35 | 54 | 88.2 | | | | | | |
| | STD2_RS012-001 | 1.5 ul | 100 | 14.71 | 96 | 130.2 | | | | | | |
| 2 | Redox | 150 ul | | Zero Point | −17.7 | 0 | | | | | | |
| | unknown | 1.5 ul | 100 | 0 | 19.8 | 37.5 | 7.4 | 13.6 | 13.5 | 99.4% | | |
| | STD1_RS12-001 | 1.5 ul | 100 | 7.35 | 63 | 80.7 | | | | | | |
| | STD2_RS012-001 | 1.5 ul | 100 | 14.71 | 97.3 | 115 | | | | | | |
| 3 | Redox | 150 ul | | Zero Point | 3 | 0 | | | | | | |
| | unknown | 1.5 ul | 100 | 0 | 27.2 | 24.2 | 8.43 | 13.6 | 11.9 | 87.3% | | |
| | STD1_RS012-001 | 1.5 ul | 100 | 7.35 | 51.8 | 48.8 | | | | | | |
| | STD2_RS012-001 | 1.5 ul | 100 | 14.71 | 71 | 68 | | | | | | |

TABLE 9
Results of testing Tysabri formulated in 10 mM Sodium Phosphate, 140 mM NaCI, pH 6.1.

| | | | | | | | | | Found % | | | |
|---|---|---|---|---|---|---|---|---|---|---|---|---|
| | | | | Tysabri | | | Un- | | Oxidation = | | Average % | |
| | | | | (μg)/% | | | known = | Expected % | Tysabri(ug)/ | Accu- | Oxidation | % |
| No. | Sample | Δ μl | Δ μg | Oxidation | E | ΔE | X | Oxidation | X | racy | (n = 3) | RSD |
| 1 | Redox | 150 μl | | Zero Point | −54.3 | 0 | | | | | 1.1% | 15.7 |
| | Unknown | 3.9 | 100 | 0 | −9.6 | 44.7 | 86.36 | 1.1 | 1.2 | 105.3% | | |
| | STD 1 | 3.9 | 100 | 90.91 | 51.1 | 105.4 | | | | | | |
| | NB-11434-113 | | | | | | | | | | | |
| | STD 2 | 3.9 | 100 | 181.82 | 91.6 | 145.9 | | | | | | |
| | NB-11434-113 | | | | | | | | | | | |
| 2 | Redox | 150 μl | | Zero Point | −37 | 0 | | | | | | |
| | Unknown | 3.9 | 100 | 0 | 2.2 | 39.2 | 86.87 | 1.1 | 1.2 | 104.7% | | |
| | STD 1 | 3.9 | 100 | 90.91 | 52.8 | 89.8 | | | | | | |
| | NB-11434-113 | | | | | | | | | | | |
| | STD 2 | 3.9 | 100 | 181.82 | 89.2 | 126.2 | | | | | | |
| | NB-11434-113 | | | | | | | | | | | |
| 3 | Redox | 150 μl | | Zero Point | −50 | 0 | | | | | | |
| | Unknown | 3.9 | 100 | 0 | 4 | 54 | 110.89 | 1.1 | 0.9 | 82.0% | | |
| | STD 1 | 3.9 | 100 | 90.91 | 54 | 104 | | | | | | |
| | NB-11434-113 | | | | | | | | | | | |
| | STD 2 | 3.9 | 100 | 181.82 | 95 | 145 | | | | | | |
| | NB-11434-113 | | | | | | | | | | | |

TABLE 10
Results of testing BART formulated in 10 mM Sodium Citrate, 150 mM L-Arginine, 0.05% PS 80, pH 6.3.

| | | Conc. | | | | | | | | Found % | | | |
|---|---|---|---|---|---|---|---|---|---|---|---|---|---|
| | | after | | | BART | | | Un- | | Oxidation = | | Average % | |
| | | buffer | | | (μg)/% | | | known = | Expected % | Bart(μg)/ | Accu- | Oxidation | % |
| No. | Sample | exchange | Δ ul | Δ μl | Oxidation | E | ΔE | X | Oxidation | X | racy | (n = 3) | RSD |
| 1 | Redox | | 150 | | | −28 | | | | | | 6.8% | 9.7 |
| | FB-BART | | 1.6 | | | −8 | 20 | | | | | | |
| | 1:20 & BE in | | | | | | | | | | | | |
| | FB_STX-100 | | | | | | | | | | | | |
| | unknown | 61 | 1.6 | 100 | 0 | 24 | 32 | 16.53 | 7.2 | 6.1 | 84.0% | | |
| | Buffer | | | | | | | | | | | | |
| | Exchanged | | | | | | | | | | | | |
| | STD1 | 61 | 1.6 | 100 | 13.89 | 61 | 69 | | | | | | |
| | (17506-7-1) | | | | | | | | | | | | |
| | 1:20 + BE | | | | | | | | | | | | |
| | STD2 | 61 | 1.6 | 100 | 27.78 | 82.2 | 90.2 | | | | | | |
| | (17506-7-1) | | | | | | | | | | | | |
| | 1:20 + BE | | | | | | | | | | | | |
| 2 | Redox | | 150 | | | −24.5 | | | | | | | |
| | FB-BART | | 1.6 | | | −11.7 | 12.8 | | | | | | |
| | 1:20 & BE in | | | | | | | | | | | | |
| | FB_STX-100 | | | | | | | | | | | | |
| | unknown | 61 | 1.6 | 100 | 0 | 12.6 | 24.3 | 13.8 | 7.2 | 7.3 | 100.6% | | |
| | Buffer | | | | | | | | | | | | |
| | Exchanged | | | | | | | | | | | | |
| | STD1 | 61 | 1.6 | 100 | 13.89 | 44.2 | 55.9 | | | | | | |
| | (17506-7-1) | | | | | | | | | | | | |
| | 1:20 + BE | | | | | | | | | | | | |
| | STD2 | 61 | 1.6 | 100 | 27.78 | 65.1 | 76.8 | | | | | | |
| | (17506-7-1) | | | | | | | | | | | | |
| | 1:20 + BE | | | | | | | | | | | | |
| 3 | Redox | | 150 | | | −45.5 | | | | | | | |
| | FB-BART | | 1.6 | | | −33.6 | 11.9 | | | | | | |
| | 1:20 & BE in | | | | | | | | | | | | |
| | FB_STX-100 | | | | | | | | | | | | |
| | unknown | 61 | 1.6 | 100 | 0 | −7.5 | 26.1 | 13.82 | 7.2 | 7.2 | 100.5% | | |
| | Buffer | | | | | | | | | | | | |
| | Exchanged | | | | | | | | | | | | |
| | STD1 | 61 | 1.6 | 100 | 13.89 | 23 | 56.6 | | | | | | |
| | (17506-7-1) | | | | | | | | | | | | |
| | 1:20 + BE | | | | | | | | | | | | |
| | STD2 | 61 | 1.6 | 100 | 27.78 | 47.1 | 80.7 | | | | | | |
| | (17506-7-1) | | | | | | | | | | | | |
| | 1:20 + BE | | | | | | | | | | | | |

TABLE 11
Results of testing Tweak formulated in 10 mM Sodium Succinate, 150 mM L-Arginine, pH 5.5.

| | | Conc. | | | | | | | | Found % | | | |
|---|---|---|---|---|---|---|---|---|---|---|---|---|---|
| | | after | | | Tweak | | | Un- | | Oxidation = | | Average % | |
| | | buffer | | | (ug)/% | | | known = | Expected % | Tweak(ug)/ | Accu- | Oxidation | % |
| No. | Sample | exchange | Δ ul | Δ ug | Oxidation | E | ΔE | X | Oxidation | X | racy | (n = 3) | RSD |
| 1 | Redox | | 150 | | | −34 | | | | | | 3.2% | 24.4 |
| | FB_Tweak | | 2.3 | | | 19.5 | 14.5 | | | | | | |
| | 1:20 & BE in | | | | | | | | | | | | |
| | FB_STX-100 | | | | | | | | | | | | |
| | unknown | 42.75 | 2.3 | 100 | 0 | 10.1 | 29.6 | 24.16 | 3.7 | 4.1 | 111.9% | | |
| | Buffer | | | | | | | | | | | | |
| | Exchange | | | | | | | | | | | | |
| | STD1 | 42.75 | 2.3 | 100 | 27.03 | 51.1 | 70.6 | | | | | | |
| | (RS030-002) | | | | | | | | | | | | |
| | 1:20 + BE | | | | | | | | | | | | |
| | STD2 | 42.75 | 2.3 | 100 | 54.05 | 80.6 | 100.1 | | | | | | |
| | (RS030-002) | | | | | | | | | | | | |
| | 1:20 + BE | | | | | | | | | | | | |
| 2 | Redox | | 150 | | | −22 | | | | | | | |
| | FB_Tweak | | 2.3 | | | −7.2 | 14.8 | | | | | | |
| | 1:20 & BE in | | | | | | | | | | | | |
| | FB_STX-100 | | | | | | | | | | | | |
| | unknown | 42.75 | 2.3 | 100 | 0 | 23 | 30.2 | 37.68 | 3.7 | 2.7 | 71.7% | | |
| | Buffer | | | | | | | | | | | | |
| | Exchange | | | | | | | | | | | | |
| | STD1 | 42.75 | 2.3 | 100 | 27.03 | 49 | 56.2 | | | | | | |
| | (RS030-002) | | | | | | | | | | | | |
| | 1:20 + BE | | | | | | | | | | | | |
| | STD2 | 42.75 | 2.3 | 100 | 54.05 | 68 | 75.2 | | | | | | |
| | (RS030-002) | | | | | | | | | | | | |
| | 1:20 + BE | | | | | | | | | | | | |
| 3 | Redox | | 150 | | | −8.5 | | | | | | | |
| | FB_Tweak | | 2.3 | | | 5.7 | 14.2 | | | | | | |
| | 1:20 & BE in | | | | | | | | | | | | |
| | FB_STX-100 | | | | | | | | | | | | |
| | unknown | 42.75 | 2.3 | 100 | 0 | 25.4 | 19.7 | 36.27 | 3.7 | 2.8 | 74.5% | | |
| | Buffer | | | | | | | | | | | | |
| | Exchange | | | | | | | | | | | | |
| | STD1 | 42.75 | 2.3 | 100 | 27.03 | 44.2 | 38.5 | | | | | | |
| | (RS030-002) | | | | | | | | | | | | |
| | 1:20 + BE | | | | | | | | | | | | |
| | STD2 | 42.75 | 2.3 | 100 | 54.05 | 56.4 | 50.7 | | | | | | |
| | (RS030-002) | | | | | | | | | | | | |
| | 1:20 + BE | | | | | | | | | | | | |

TABLE 12
Results of testing Anti-BDCA2 formulated in 10 mM Sodium Succinate, 150 mM L-Arginine HCl, 0.05% PS80, pH 6.0.

| | | Conc. | | | | | | | | Found % | | | |
|---|---|---|---|---|---|---|---|---|---|---|---|---|---|
| | | after | | | BDCA2 | | | Un- | | Oxidation = | | Average % | |
| | | buffer | | | (ug)/% | | | known = | Expected % | BDCA2(ug)/ | Accu- | Oxidation | % |
| No. | Sample | exchange | Δ ul | Δ ug | Oxidation | E | ΔE | X | Oxidation | X | racy | (n = 3) | RSD |
| 1 | Redox | | 150 | | | −37.1 | | | | | | 4.9% | 24.8 |
| | FB_BDC A2 | | 1.5 | | | −24.7 | 12.4 | | | | | | |
| | 1:20 & BE in | | | | | | | | | | | | |
| | FB_STX-100 | | | | | | | | | | | | |
| | unknown | 65 | 1.5 | 100 | 0 | 13.1 | 37.8 | 23.1 | 5.7 | 4.3 | 75.9% | | |
| | Buffer | | | | | | | | | | | | |
| | Exchanged | | | | | | | | | | | | |
| | STD1(175 | 65 | 1.5 | 100 | 17.45 | 44.5 | 69.2 | | | | | | |
| | 98-08) | | | | | | | | | | | | |
| | 1:20 + BE | | | | | | | | | | | | |
| | STD2(175 | 65 | 1.5 | 100 | 35.09 | 71.6 | 96.3 | | | | | | |
| | 98-08) | | | | | | | | | | | | |
| | 1:20 + BE | | | | | | | | | | | | |
| 2 | Redox | | 150 | | | −21.2 | | | | | | | |
| | FB_BDC A2 | | 1.5 | | | −14.1 | 7.1 | | | | | | |
| | 1:20 & BE in | | | | | | | | | | | | |
| | FB_STX-100 | | | | | | | | | | | | |
| | unknown | 65 | 1.5 | 100 | 0 | 12.6 | 26.7 | 15.95 | 5.7 | 6.3 | 110.0% | | |
| | Buffer | | | | | | | | | | | | |
| | Exchanged | | | | | | | | | | | | |
| | STD1(175 | 65 | 1.5 | 100 | 17.54 | 40.6 | 54.7 | | | | | | |
| | 98-08) | | | | | | | | | | | | |
| | 1:20 + BE | | | | | | | | | | | | |
| | STD2(175 | 65 | 1.5 | 100 | 35.09 | 70.6 | 84.7 | | | | | | |
| | 98-08) | | | | | | | | | | | | |
| | 1:20 + BE | | | | | | | | | | | | |
| 3 | Redox | | 150 | | | −16.8 | | | | | | | |
| | FB_BDC A2 | | 1.5 | | | 0.4 | 17.2 | | | | | | |
| | 1:20 & BE in | | | | | | | | | | | | |
| | FB_STX-100 | | | | | | | | | | | | |
| | unknown | 65 | 1.5 | 100 | 0 | 28 | 27.6 | 24.23 | 5.7 | 4.1 | 72.4% | | |
| | Buffer | | | | | | | | | | | | |
| | Exchanged | | | | | | | | | | | | |
| | STD1(175 | 65 | 1.5 | 100 | 17.54 | 53.2 | 52.8 | | | | | | |
| | 98-08) | | | | | | | | | | | | |
| | 1:20 + BE | | | | | | | | | | | | |
| | STD2(175 | 65 | 1.5 | 100 | 35.09 | 70 | 69.6 | | | | | | |
| | 98-08) | | | | | | | | | | | | |
| | 1:20 + BE | | | | | | | | | | | | |

TABLE 13
Results of testing Daclizumab formulated in 40 mM Succinate, 100 mM Sodium Chloride, pH 6.0.

| | | | | | | | | | Found % | | | |
|---|---|---|---|---|---|---|---|---|---|---|---|---|
| | | | | DAC | | | Un- | | Oxidation = | | Average % | |
| | | | | (ug)/% | | | known = | Expected % | DAC(ug)/ | Accu- | Oxidation | % |
| No. | Sample | Δ ul | Δ ug | Oxidation | E | ΔE | X | Oxidation | X | racy | (n = 3) | RSD |
| 1 | Redox | 150 ul | | | −50.20 | | | | | | 1.7% | 11.8 |
| | FB_DAC | 1.5 | | Zero Point | −38.80 | | | | | | | |
| | DAC | 0.7 | 100 | 0.00 | −3.10 | 35.70 | 60.30 | 1.53 | 1.7 | 108.4% | | |
| | unknown % | | | | | | | | | | | |
| | Oxidation | | | | | | | | | | | |
| | STD1 | 0.7 | 100 | 65.36 | 42.4 | 81.20 | −60.30 | | | | | |
| | 17199-51-4 | | | | | | | | | | | |
| | (150 mg/ml) | | | | | | | | | | | |
| | STD2 | 0.7 | 100 | 130.72 | 77.90 | 116.70 | | | | | | |
| | 17199-51-4 | | | | | | | | | | | |
| | (150 mg/ml) | | | | | | | | | | | |
| 2 | Redox | 150 ul | | | −43.00 | | | | | | | |
| | FB_DAC | 1.5 | | Zero Point | −30.50 | | | | | | | |
| | DAC | 0.7 | 100 | 0.00 | −2.70 | 27.80 | 53.62 | 1.53 | 1.9 | 121.9% | | |
| | unknown % | | | | | | | | | | | |
| | Oxidation | | | | | | | | | | | |
| | STD1 | 0.7 | 100 | 65.36 | 43 | 73.50 | −53.62 | | | | | |
| | 17199-51-4 | | | | | | | | | | | |
| | (150 mg/ml) | | | | | | | | | | | |
| | STD2 | 0.7 | 100 | 130.72 | 71.90 | 102.40 | | | | | | |
| | 17199-51-4 | | | | | | | | | | | |
| | (150 mg/ml) | | | | | | | | | | | |
| 3 | Redox | 150 ul | | | −32.00 | | | | | | | |
| | FB_DAC | 1.5 | | Zero Point | −20.70 | | | | | | | |
| | DAC | 0.7 | 100 | 0.00 | 5.40 | 26.10 | 65.72 | 1.53 | 1.5 | 99.5% | | |
| | unknown % | | | | | | | | | | | |
| | Oxidation | | | | | | | | | | | |
| | STD1 | 0.7 | 100 | 65.36 | 37.1 | 57.80 | −65.72 | | | | | |
| | 17199-51-4 | | | | | | | | | | | |
| | (150 mg/ml) | | | | | | | | | | | |
| | STD2 | 0.7 | 100 | 130.72 | 60.60 | 81.30 | | | | | | |
| | 17199-51-4 | | | | | | | | | | | |
| | (150 mg/ml) | | | | | | | | | | | |

TABLE 14
Summary of percent oxidation results obtained by indirect potentiometry with
10⁻²/10⁻⁴ M of Ru(NH₃)₆³⁺/Ru(NH₃)₆²⁺ mediator system

| Protein | S1 | S2 | S3 | AVERAGE | STDEV | % RSD | Expected | % Accuracy |
|---|---|---|---|---|---|---|---|---|
| STX-100 | 14.6 | 13.5 | 11.9 | 13.3 | 1.4 | 10.2 | 13.6 | 98.0 |
| Tysabri | 1.2 | 1.2 | 1.1 | 1.2 | 0.1 | 4.9 | 1.1 | 106.1 |
| Tweak | 4.1 | 2.7 | 2.8 | 3.2 | 0.8 | 24.4 | 3.7 | 86.5 |
| aBDC2 | 4.3 | 6.3 | 4.1 | 4.9 | 1.2 | 24.8 | 5.7 | 86.0 |
| DAC | 1.7 | 1.9 | 1.5 | 1.7 | 0.2 | 11.8 | 1.5 | 113.3 |

### Example 4: Stability of STX-100

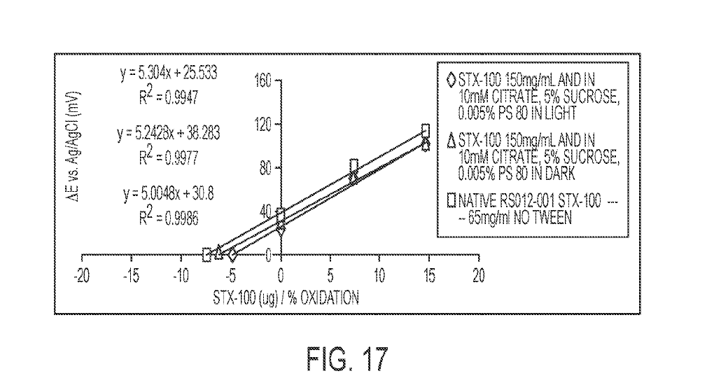

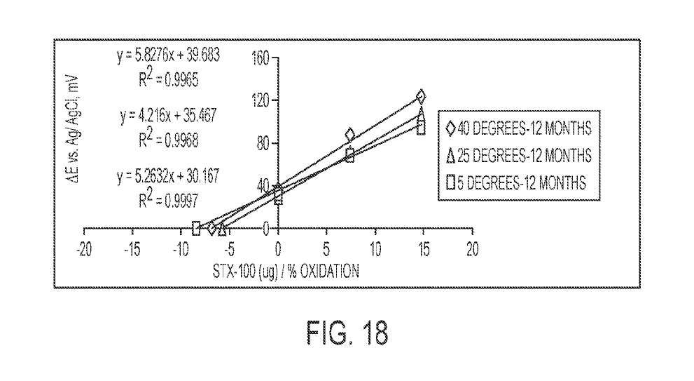

Stability of STX-100 was evaluated potentiometrically using the methods disclosed in Examples 1-3. The % oxidation of a panel of STX-100 was determined using indirect potentiometry including a reference standard where the % oxidation of the reference standard was determined by mass spectrometry. The redox couple used for each experiment was 10⁻² M/10⁻⁴ M of Ru(NH₃)₆³⁺/Ru(NH₃)₆²⁺. Each experiment was performed in triplicate. Results obtained were compared with different environmental conditions, including exposure to light, temperature, and the effects of different formulations. FIG. 17 and Table 15 illustrate the effect of light exposure on STX-100. FIG. 18 and Table 16 illustrate the effect of temperature exposure on STX-100.

TABLE 15
Stability Study on Light-Stressed STX-100

| | | | STX-100 | | | | | Found % |
|---|---|---|---|---|---|---|---|---|
| | | | (ug)/% | | | Unknown = | STD % | Oxidation = |
| Sample | Δ μl | Δ μg | Oxidation | E | ΔE | X | Oxidation | STX 100 μg/X |
| STX-100 150 | 0.7 | 100 | 0.00 | −18.00 | 23.20 | 4.81 | 13.6 | 20.8% |
| mg/mL and in 10 | | | | | | | | |
| mM citrate, 5% | | | | | | | | |
| sucrose, 0.005% | | | | | | | | |
| PS80 in LIGHT | | | | | | | | |
| STX-100 150 | 0.7 | 100 | 0.00 | 1.40 | 19.40 | 6.15 | 13.6 | 16.3% |
| mg/mL and in 10 | | | | | | | | |
| mM citrate, 5% | | | | | | | | |
| sucrose, 0.005% | | | | | | | | |
| PS80 in DARK | | | | | | | | |
| Non Stressed | 1.5 | 100 | 0.00 | −8.40 | −9.50 | 7.30 | 13.6 | 13.7% |
| RS012-001 | | | | | | | | |
| STX-100 | | | | | | | | |
| 65 mg/ml | | | | | | | | |
| No Tween | | | | | | | | |

TABLE 16
Stability Study Results on Temperature-Stressed STX-100, 14 Months Study

| | | | STX-100 | | | | | Found % |
|---|---|---|---|---|---|---|---|---|
| | | | (ug)/% | | | Unknown = | STD % | Oxidation = |
| | Δ ul | Δ ug | Oxidation | E | ΔE | X | Oxidation | STX-100(ug)/X |
| Sample 1 (40 | | | | | | | | |
| Degrees 14 Months) | | | | | | | | |
| Redox | 150 | 100 | | −41.50 | | | | |
| Unknown Stressed | 1.0 | 100 | 0.00 | −4.10 | 37.40 | 6.81 | 13.6 | 14.7% |
| STX-100 Buffer | | | | | | | | |
| Exchanged | | | | | | | | |
| STD 1 (RS012-001) | 1.0 | 100 | 7.35 | 45.6 | 87.10 | | | |
| 100 ug | | | | | | | | |
| STD 2 (RS012-001) | 1.0 | 100 | 14.71 | 81.6 | 123.10 | | | |
| 100 ug | | | | | | | | |
| Sample 2 (25 | | | | | | | | |
| Degrees 14 Months) | | | | | | | | |
| Redox | 150 | 100 | | −33.00 | | | | |
| Unknown Stressed | 1.0 | 100 | 0.00 | −3.40 | 29.60 | 5.73 | 13.6 | 17.5% |
| STX-100 Buffer | | | | | | | | |
| Exchanged | | | | | | | | |
| STD 1 (RS012-001) | 1.0 | 100 | 7.35 | 37 | 70.00 | | | |
| 100 ug | | | | | | | | |
| STD 2 (RS012-001) | 1.0 | 100 | 14.71 | 74 | 107.00 | | | |
| 100 ug | | | | | | | | |
| Sample 1 (5 Degrees | | | | | | | | |
| 14 Months) | | | | | | | | |
| Redox | 150 | 100 | | −18.80 | | | | |
| Unknown Stressed | 1.0 | 100 | 0.00 | 15.00 | 33.80 | 8.41 | 13.6 | 11.9% |
| STX-100 Buffer | | | | | | | | |
| Exchanged | | | | | | | | |
| STD 1 (RS012-001) | 1.0 | 100 | 7.35 | 51 | 69.80 | | | |
| 100 ug | | | | | | | | |
| STD 2 (RS012-001) | 1.0 | 100 | 14.71 | 77 | 95.80 | | | |
| 100 ug | | | | | | | | |

Methods provided herein enable rapid assessment of protein oxidation status. The analysis is expedited from 24-36 hours by mass spectrometry to 15-30 min by indirect potentiometry for multiple samples. This outcome relates in part due to application of “mediator” approach and microelectrodes. The instrumentation for indirect potentiometric measurements is inexpensive and portable, and the method allows for low samples consumption. The approach can be configured as an automated system for high-throughput screening of oxidative protein modifications.

Indirect potentiometric methods provided herein are useful with a wide variety of applications, ranging from protein oxidation control to monitoring oxidation levels in water-soluble raw materials composed of Tween. Tween 20 and/or Tween 80 are used in the formulation of therapeutic proteins. The diagnostic value and positive impact of understanding the impact of Tween oxidation on protein stability and oxidation is useful in the context of biotherapeutic protein production.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of assessing a pharmaceutical preparation, the method comprising:
determining a redox potential of the pharmaceutical preparation; and
determining the oxidation status of the pharmaceutical preparation based on the redox potential of the pharmaceutical preparation.

2. The method of claim 1, wherein the step of determining the redox potential of the pharmaceutical preparation comprises determining a potential of a redox couple corrected by a formulation buffer of the pharmaceutical preparation, and wherein the oxidation status of the pharmaceutical preparation is determined based on a change in potential of the redox couple due to its interaction with the pharmaceutical preparation.

3. The method of claim 1 further comprising
(i) determining a potential between an indicator electrode and a reference electrode disposed in a solution that comprises a redox couple, thereby determining the redox potential;
(ii) determining a change in potential between the indicator electrode and the reference electrode resulting from presence of the pharmaceutical preparation in the solution that comprises the redox couple; and
(iii) determining the oxidation status of the pharmaceutical preparation based on the change in potential.

4. The method of claim 1 further comprising
(i) determining a potential between an indicator electrode and a reference electrode disposed in a solution that comprises a redox couple, thereby determining the redox potential;
(ii) determining a potential between an indicator electrode and a reference electrode resulting from presence of the formulation buffer in a solution that comprises a redox couple, thereby correcting the redox potential;
(iii) determining a change in potential between the indicator electrode and the reference electrode resulting from presence of the pharmaceutical preparation in the solution that comprises the redox couple and formulation buffer; and
(iv) determining the oxidation status of the pharmaceutical preparation based on the change in potential.

5. The method of claim 4, wherein step (iv) comprises evaluating a reference standard that relates a change in potential to an oxidation status of the pharmaceutical preparation.

6. The method of claim 5, wherein the reference standard relates the change in potential to a mass spectroscopically determined oxidation status of the pharmaceutical preparation.

7. The method of claim 1, wherein the indicator electrode is a platinum, gold, palladium, rhodium, or carbon electrode.

8. The method of claim 7, wherein the reference electrode is a silver/silver chloride reference electrode.

9. The method of any one of claims 1 to 8, wherein the redox couple comprises Hexamamineruthenium(II)chloride and Hexamamineruthenium(III)chloride.

10. The method of claim 9, wherein the concentration of Hexamamineruthenium(II)chloride is between 10⁻² M and 10⁻⁴ M.

11. The method of claim 9 or 10, wherein the concentration of Hexamamineruthenium(III)chloride is between 10⁻² M and 10⁻⁴ M.

12. The method in any one of claims 9 to 11, wherein the concentration of Hexamamineruthenium(II)chloride is 10⁻² M and the concentration of Hexamamineruthenium(III)chloride is 10⁻⁴ M.

13. The method of any one of claims 1 to 12, wherein the pharmaceutical preparation comprises a protein, a nucleic acid, or a small molecule.

14. The method of any one of claims 1 to 13, wherein the pharmaceutical preparation comprises a protein.

15. The method of claim 14, wherein the protein is an immunoglobulin or fragment thereof.

16. The method in any of claims 1 to 15, wherein the pharmaceutical preparation comprises STX-100, natalizumab, BIIB037, Anti-TWEAK, Anti-BDCA2, Daclizumab.

17. The method in any one of claims 1 to 16, wherein the pharmaceutical preparation comprises an excipient.

18. The method of claim 17, wherein the excipient comprises a polysorbate or an amino acid.

19. The method of claim 18, wherein the polysorbate is selected from the group consisting of polysorbate 20, polysorbate 40, polysorbate 60, and polysorbate 80.

20. The method of claim 18, wherein the amino acid comprises arginine, glycine or histidine.

21. The method of any one of claims 1 to 20, wherein the change in potential measured in step (ii) is adjusted based on an extent of change in potential between the indicator electrode and the reference electrode that results from a buffer component of the pharmaceutical preparation.

22. The method of claim 21, wherein the buffer component comprises sodium sulfate, sodium citrate, sodium phosphate, succinate, sodium chloride, potassium nitrate, or sucrose.

23. The method of any one of claims 1 to 21 further comprising, prior to step (i), removing a component of the pharmaceutical preparation that affects the potential of the redox couple.

24. The method of claim 23, wherein the component is an arginine or a polysorbate.

25. The method of any one of claims 1 to 24, wherein the oxidation status is indicative of activity of the pharmaceutical preparation.

26. The method of 25 further comprising evaluating activity of the pharmaceutical preparation using a bioassay.

27. The method in any one of claims 25 to 26, wherein the bioassay comprises evaluating efficacy or toxicity of the pharmaceutical preparation.

28. The method in any one of claims 1 to 27, wherein the oxidation status of the pharmaceutical preparation is determined on two or more occasions.

29. The method of 28, further comprising determining a change in activity of the pharmaceutical preparation based on the oxidation status of the pharmaceutical preparation is determined on the two or more occasions.

30. A method of assessing a pharmaceutical preparation, the method comprising:
potentiometrically determining the oxidation status of the pharmaceutical preparation.

31. A method of assessing activity of a biomolecule, the method comprising
determining a redox potential between an indicator electrode and a reference electrode disposed in a solution comprising the biomolecule; and
determining activity of the biomolecule based on the redox potential.

32. A method of assessing the activity of a biomolecule, the method comprising
(i) determining a potential between an indicator electrode and an reference electrode disposed in a solution that comprises a redox couple;
(ii) determining a change in potential between the indicator electrode and the reference electrode resulting from presence of the biomolecule in the solution that comprises the redox couple; and
(iii) determining the activity of the biomolecule based on the change in potential.

33. The method of claim 32, wherein step (iii) comprises evaluating a reference standard that relates a change in potential to the activity of the biomolecule.

34. The method in any one of claims 1 to 33, wherein the biomolecule is a protein, nucleic acid, or small molecule.

35. The method of claim 34, wherein the protein is an immunoglobulin or fragment thereof.

36. The method in any one of claims 1 to 35, wherein the indicator electrode is a platinum, gold, palladium, rhodium, or carbon electrode.

37. The method of any one of claims 1 to 36, wherein the reference electrode is a silver/silver chloride or saturated calomel reference electrode.

38. The method of any one of claims 1 to 37, wherein the redox couple comprises Hexamamineruthenium(III)chloride and Hexamamineruthenium(II)chloride.

39. The method of claim 38, wherein the concentration of Hexamamineruthenium(II)chloride is between 10⁻² M and 10⁻⁴ M.

40. The method of any one of claims 38 to 39, wherein the concentration of Hexamamineruthenium(III)chloride is between 10⁻² M and 10⁻⁴ M.

41. The method in any one of claims 38 to 40, wherein the concentration of Hexamamineruthenium(II)chloride is 10⁻² M and the concentration of Hexamamineruthenium(III)chloride is 10⁻⁴ M.

42. A system for assessing the oxidation status of a pharmaceutical preparation, the system comprising
(i) a container configured for housing a solution;
(ii) an indicator electrode and a reference electrode, both disposable in the container;
(iii) a potential measuring device operably connectable to the indicator electrode and reference electrode and configured for obtaining one or more potential measurements between the indicator electrode and the reference electrode; and
(iv) a computer operably connectable to the potential measure device and configured for determining the oxidation status of the pharmaceutical preparation based on one or more potential measurements obtained from the potential measuring device while the pharmaceutical preparation is present in the solution.

43. The system of claim 42, wherein the solution comprises a redox couple, and wherein the computer is configured for determining the oxidation status of the pharmaceutical preparation based on a change in potential between the indicator electrode and the reference electrode resulting from presence of the pharmaceutical preparation in the solution that comprises the redox couple.

44. The system of claim 42, wherein the computer comprises:
an input interface configured to receive information from the potential measuring device indicative of one or more potential differences measured between the indicator electrode and reference electrode;
at least one processor programmed to evaluate a model that relates the one or more potential differences to the oxidation status of the pharmaceutical preparation, and
an output interface configured to output a signal indicative of the oxidation status.

45. The system of claim 44, wherein the computer is configured to determine the oxidation status of a protein based on a reference standard that relates potential differences to a mass spectrometrically determined oxidation status of the pharmaceutical preparation.
